(19) United States
(12) Patent Application Publication
(10) Pub. No.: US 2015/0064697 A1
(43) Pub. Date: Mar. 5, 2015

(54) P53 COOPERATION WITH DNA METHYLATION AND SUICIDAL INTERFERON RESPONSE TO CONTROL SILENCING OF REPEATS AND NCRNAS

(71) Applicant: Health Research, Inc., Buffalo, NY (US)

(72) Inventors: Andrei Gudkov, East Aurora, NY (US); Yekaterina Leonova, East Aurora, NY (US)

(21) Appl. No.: 14/388,297

(22) PCT Filed: Mar. 29, 2013
(86) PCT No.: PCT/US13/34632

§ 371 Date: Sep. 26, 2014

Related U.S. Application Data
(60) Provisional application No. 61/617,394, filed on Mar. 29, 2012.

(51) Int. Cl. C12Q 1/68

(52) U.S. Cl. CPC C12Q 1/6886; C12Q 2600/154
USPC 435/6.11

(57) ABSTRACT

Provided is a disclosure of methods involving determining RNA transcripts transcribed from repeats of polynucleotide sequences, the transcription of which are normally repressed by a combination of functional p53 and DNA methylation, and methods of detecting interferon, and determining constitutive activation of an interferon type I response. The methods permit identification of cancerous or precancerous cells, can identify non-functional p53, and provide therapeutic and prophylactic approaches for cancer.

### CROSS REFERENCE TO RELATED APPLICATIONS

This application claims priority to U.S. Provisional application No. 61/617,394, filed on Mar. 29, 2012, the disclosure of which is incorporated herein by reference.

### FIELD

The present invention relates generally to detecting and treating cancer and/or pre-cancerous conditions.

### BACKGROUND

Mammalian genomes are abundant in noncoding DNA sequences whose biological function is underexplored. This portion of the genome includes DNA transposons, long-terminal repeat (LTR) retrotransposons, long interspersed nuclear elements (LINEs) and short interspersed nuclear elements (SINEs). They all are evolutionary younger than structural genes and originate from reverse transcription products of either endogenous viruses (LINEs) or certain RNA pol III-driven transcripts (SINEs)¹. These elements vary in length and copy numbers. DNA transposons are remnants of ancient elements appear throughout modern genome least frequently and it is improbable that any are transpositionally active^{2,3}. Contrariwise, LINEs, and by virtue SINEs that utilize the reverse transcriptional machinery provided by LINEs, have both shown to retain some ability to retrotranspose⁴. Additional to interspersed elements, the mammalian genome also contains tandemly organized repeats that are commonly known as satellite DNAs⁵. They are highly reiterated sequences that are the main component of the centromeric and pericentromeric heterochromatin^{6,7} and are considered important and evolutionary conserved structural elements of the chromosome⁸. Altogether, these repeats occupy more than 50% of human or mouse genome⁹.

The analysis of the genomic content of retroelements within the genomes of different mammalian species suggests that they accumulated through multiple bursts of “explosions”, where the periods of rapid amplification are followed by dormancy^{10,11}. Each act of amplification of retroelements, especially most abundant SINEs, was associated with extensive insertional mutagenesis presumably contributing to diversity of mammalian species. Explosive nature of multiplication of SINEs and the fact that they all are located in intergenic areas, pseudogenes and noncoding regions of genes suggest that their amplifications were series of genetic catastrophies associated with massive loss of those genomes which acquired inserts of SINEs inside essential genes¹². The genome contact with SINEs (and potentially other transposable elements) resembles a host-parasite coevolution, since integration of new SINE copies into coding or regulatory sequences disturbs the gene expression^{1,13}. This idea is supported by remarkable genus specificity of retroelements and chronological coincidence of their explosions with appearance of major bifurcations of mammalian evolutionary tree.

Given the abundance and genomic distribution of these elements, both their re-integration and expression could lead to detrimentally unstable conditions within the genome. It would be logical to presume that maintenance of genetic stability of current species depends on their ability to suppress amplification of tandem and dispersed repetitive elements, in general, and SINEs, in particular. In fact, these sequences are not normally actively transcribed as distinct elements but rather as integral parts of non-coding regions of larger protein-coding transcripts. SINEs are particularly abundant in introns, and many mammalian genes contain SINE copies. They also occur within the 3′-untranslated regions of the exons¹². Vast majority of SINEs are located in heavily methylated regions of DNA, which is believed to be one of the mechanisms of their epigenetic silencing¹⁴. However, in mammalian cells, DNA methylation commonly occurs as a consequence of epigenetic silencing occurring at chromatin level¹⁵. If there is such mechanism that suppresses transcription of SINEs and other retroelements, it remains unknown.

The majority of these elements is located in “deserted” regions of genome and is normally silent. However, under certain circumstances both retroelements and some classes of satellite DNA can be transcribed. Although, events underlying and triggering such activation are not well understood, they include rapid and dramatic increase in response to heat shock^{16,17}, viral infections¹⁸, various DNA damaging cell stresses^{19,20} and translational inhibition²¹; all of each have highlighted the possibility that these transcripts serve a physiological role within the organism. Additionally, transcription of both is frequently observed in tumors in a variety of models of mouse and human origin^{22,23}. Mechanisms underlying transcriptional activation of normally “silent” repeats in stressed and transformed cells and physiological consequences of this activation remain poorly understood. Therefore, there is an ongoing an unmet need for compositions and methods related to prophylaxis, therapy, diagnostics and drug discovery in the area related to activation of non-coding elements of the genome.

### SUMMARY

The present disclosure relates generally in one aspect to analyzing biological samples for the evidence of a process referred to herein as “TRAIN” which is a term we developed to represent our discovery that “transcription of repeats activates interferon.” Without intending to be constrained by any particular theory, and as described further below, it is considered that TRAIN manifests itself at least in part because of reduced or absent functional p53 activity, which may also occur with concomitant irregular DNA methylation. By “irregular” it is meant that the DNA methylation (or demethylation) of a DNA segment, such as gene segment, such as a promoter or coding region, or any other segment of DNA that affects transcription, has a methylation pattern that is typically found in a non-normal cell, such as a pre-cancerous or cancer cell, but not in non-cancer cells.

The present disclosure includes but is not necessarily limited to the discovery of massive transcription of major classes of short interspersed repeats (SINEs B1 and B2), both strands of near-centromeric satellite DNAs containing tandem repeats (major γ-satellites), and multiple species of non-coding RNAs in p53-deficient, but not in p53-wild type, mouse fibroblasts treated with the DNA demethylating agent 5-aza-2′-deoxycytidine. The abundance of these new transcripts exceeded the level of β-actin mRNA by over 150-fold. Accumulation of these transcripts, which are capable of forming double-stranded RNA, was accompanied by a strong, endogenous, apoptosis-inducing type I interferon (IFN) response. This TRAIN process was observed in spontaneous tumors in two models of cancer-prone mice, presumably reflecting naturally occurring DNA hypomethylation and p53 inactivation in cancer. These discoveries indicate that p53 and IFN cooperate to prevent accumulation of cells with activated repeats and provide an explanation for the deregulation of IFN function frequently seen in tumors.

Approaches of the present disclosure in various and non-limiting embodiments involve testing biological samples for indicia of the presence or absence of TRAIN. In embodiments, the indicia of TRAIN may include RNA transcripts transcribed from repeats of polynucleotide sequences, the transcription of which is normally repressed by a combination of functional p53 and DNA methylation, and/or constitutively active type I interferon responses. In embodiments, indicia of TRAIN can include the presence or absence of polynucleotides, or amounts of polynucleotides that are compared to a reference, wherein the polynucleotides comprise RNA polynucleotides. The RNA polynucleotides can comprise ncRNA species and/or RNA transcripts transcribed from polynucleotide sequence repeats, the transcription of which is normally suppressed by functional p53. In embodiments, the RNA polynucleotides have sequences that are described by the GenBank accession numbers shown in Tables 1 and 2 and 3, and for which each sequence associated with each GenBank accession numbers is incorporated herein by reference as of the earliest priority date of this application or patent. SEQ ID NO:s are also provided for the targets identified in Tables 1, 2 and 3. The invention also comprises detecting and/or quantitating any of the sequences disclosed herein from SEQ ID NO:276 to SEQ ID NO:351, inclusive, and including all SEQ ID NO:s there between. It will be recognized that the polynucleotide sequences associated with the GenBank accession and the SEQ ID NOs presented herein may be DNA sequences, such as cDNAs. The invention includes the RNA equivalents of all such DNA sequences, meaning the disclosure includes each DNA sequence wherein each T is substituted with a U. It will also be recognized by those skilled in the art that certain embodiments described herein were developed using murine models and therefore some polynucleotide sequences are provided as murine sequences. Additionally, to identify certain sequences that we detected as being transcribed in the absence of p53 or when p53 is non-functional (such as sequences for tandem repeats (e.g., satellites) and dispersed elements and retroelements (SINEs, IAPs, LINEs, and the like) genetic databases were queried and in some cases other mammalian and non-mammalian sequences were identified, such as some of the sequences described in SEQ ID NO:276 to SEQ ID NO:351. The invention includes the human homolog and RNA equivalent of every non-human animal DNA polynucleotide sequence presented herein. Accordingly, when reference is made to any polynucleotide sequence that is, for example an RNA transcript or the cDNA of an RNA transcript of a non-human animal, the disclosure and the claims include the cDNA and RNA transcript of the human homolog. Given the benefit of the present disclosure those skilled in the art will be able to determine the sequence of the human homolog from the non-human animal sequences presented herein.

In embodiments, the present disclosure includes approaches for detection of cancer or a pre-cancerous condition in a mammal. Such approaches can comprise detecting in a sample obtained or derived from an individual a constitutively active type I interferon response, and/or RNA transcripts transcribed from repeats of polynucleotide sequences, the transcription of which is normally repressed by a combination of functional p53 and DNA methylation.

In another aspect the present disclosure provides a method for predicting tumor sensitivity to a DNA demethylating agent by determining the absence of indicia of TRAIN.

In another aspect, the disclosure provides a method for prophylaxis and/or treatment of cancer by selective elimination of cells with inactivated p53. This aspect can comprise administering to an individual in need of the prophylaxis and/or therapy a composition comprising an effective amount of one or more agents which induce DNA demethylation. In one non-limiting embodiment, the agent is 5-aza-dC.

In another aspect, the disclosure provides a method of selection and/or identification of agents, which are capable of DNA demethylation in cells with inactivated p53. This aspect can comprise contacting cells that comprise inactivated p53 (or no p53) with one or more test agents, and determining whether or not the test agents induce activation of transcription of repeats and/or ncRNA species, which are normally suppressed by functional p53.

In another aspect, the disclosure provides a method of classification of a tumor. The method can comprise determining whether or not the tumor exhibits indicia of TRAIN, and classifying the tumor as positive or negative for indicia of TRAIN.

In another aspect, the disclosure provides a method of determination of functional status of p53 in a cell or tumor. This method can comprise determining whether or not the cell or the tumor exhibits indicia of TRAIN, wherein an absence of indicia of TRAIN indicates the p53 is functional, and wherein the presence of the indicia of TRAIN indicates the p53 is not functional.

In another aspect, the disclosure provides a method of determination of tumor sensitivity to treatment with oncolytic virus therapy by detection of indicia of TRAIN in a sample obtained or derived from an individual. An absence of indicia of TRAIN indicates tumor sensitivity, and the presence of the indicia of TRAIN indicates tumor insensitivity.

In another aspect the disclosure includes a method of determination of tumor sensitivity to interferon therapy by detection of indicia of TRAIN in the tumor, wherein the presence of the indicia of TRAIN is indicative that the tumor is likely to be sensitive to interferon therapy, and wherein the absence of the indicia of TRAIN is indicative that the tumor is likely to not be sensitive to interferon therapy.

In another aspect the disclosure includes a method of detection of TRAIN comprising using in situ hybridization techniques with oligonucleotides specific for transcripts and/or ncRNA species characteristic of TRAIN.

In another aspect the disclosure includes a method detection of TRAIN in tumors and/or normal cells and/or tissues comprising using immunohistochemical assessment of ongoing interferon signaling.

In another aspect the disclosure includes a method of screening of potentially carcinogenic substances for the ability to induce TRAIN in mammalian cells.

In another aspect the disclosure includes a method of diagnostics of aging comprising determining indicia of TRAIN in a sample of normal tissue and/or and body fluids obtained or derived from a mammal.

In another aspect the disclosure includes a method of screening and/or identification of agents suitable for treatment and/or prophylaxis of aging and/or cancer by identifying agents with selective cytotoxicity against cells with activated TRAIN. In embodiments, identifying the agents comprise contacting cells with activated TRAIN with test agents, wherein a test agent which kills cells with activated TRAIN, but does not kill cells which do not exhibit activated TRAIN, is indicated to be an agent that is suitable for treatment and/or prophylaxis of aging and/or cancer.

### SUMMARY OF THE FIGURES

FIG. 1. Primary cells deficient in functional p53 are hypersensitive to 5-aza-dC treatment. (FIG. 1A) p53-WT and p53-null MEFs were treated with 0, 5 or 10 μM 5-aza-dC for 120 hours. Viable cells were visualized by methylene blue staining. (FIG. 1B) Western blot detection of p53 and β-actin (loading control) proteins in p53-WT MEFs (lanes 1-2), p53-null MEFs (lanes 3-4), and p53-WT MEFs expressing shRNA against p53 (shp53, lanes 5-6) or the p53-inactivating genetic suppressor element-56 (GSE56, lanes 7-8) left untreated (−) or treated (+) with 500 nM doxorubicin (Dox) for 16 hours. (FIG. 1C) Cytotoxicity of 5-aza-dC in cells described in (FIG. 1B). Cells were treated with the indicated concentrations of 5-aza-dC for 5 days. Viability was determined by methylene blue staining and extraction, followed by spectrophotometric quantification. Percent viability is shown relative to control cells treated with 0.1% DMSO. Here and below, error bars show standard deviations for assays performed in triplicate. (FIG. 1D) Cytotoxicity of 5-aza-dC (5 day in vitro treatment) in primary cells from different tissues of adult p53-WT and p53-null mice was determined as in (FIG. 1C). (FIG. 1E) Caspase-3,7 activity (cleavage of the fluorescent substrate AC-Devd-AMC) in p53-WT and p53-null MEFs treated with the indicated concentrations of 5-aza-dC for 48 hours or 1 μM doxorubicin (Dox) for 16 hours. (FIG. 1F) Western blot analysis of DNA methyltransferase I (DNMT-I) and β-actin (loading control) protein levels in p53-WT and p53-null MEFs treated with the indicated concentrations of 5-aza-dC for 48 hours. (FIG. 1G) The overall extent of genomic DNA methylation in p53-WT and p53-null MEFs left untreated (U) or treated (T) with 10 μM 5-aza-dC for 48 hours was determined by digestion of DNA with the methylation-sensitive restriction enzyme McrBC which only cuts its sites that are methylated.

FIG. 2. Illumina microarray-based analysis of gene expression in p53-WT and p53-null MEFs left untreated or treated with 10 μM 5-aza-dC for 48 hours. (FIG. 2A) Middle: Venn diagram showing no overlap between the 55 and 124 genes upregulated (>5-fold) by 5-aza-dC in p53-WT and p53-null cells, respectively. Left and right panels (bar graphs): mRNA expression levels (signal intensity on Illumina microarray) of genes identified as 5-aza-dC-induced in p53-WT MEFs (left panel) or in p53-null MEFs (right panel) in all four samples (p53-WT and p53-null MEFs left untreated or treated with 5-aza-dC). (FIG. 2B) Fold-induction (log scale; 5-aza-dC-treated relative to untreated) of a subset of genes identified as 5-aza-dC-induced in either p53-WT MEFs or p53-null MEFs. Note the scale of induction of IFNβ1 in p53-null MEFs. (FIG. 2C) Validation of Illumina microarray gene expression analysis was performed by RT-PCR using independently isolated RNA from p53-WT and p53-null MEFs left untreated or treated with 10 μM 5-aza-dC for 48 hours and specific primers for mouse IFNβ1, H2-Q6, IRF7 and β-actin (control).

FIG. 3. Treatment of p53-null MEFs with 5-aza-dC induces a lethal IFN response. (FIG. 3A) Western blot detection of p53 and β-actin (loading control) proteins in MEFs from ifnar^{-/-} mice expressing endogenous WT p53 (lanes 1-2) or shRNA against p53 (shp53, lanes 3-4) or the p53-inactivating genetic suppressor element-56 (GSE56, lanes 7-8) left untreated (−) or treated (+) with 500 nM doxorubicin (Dox) for 16 hours. (FIG. 3B) Cytotoxicity of 5-aza-dC in cells differing in p53 and IFNAR status. Cells were treated with the indicated concentrations of 5-aza-dC for 5 days. Viability was determined by methylene blue staining and extraction, followed by spectrophotometric quantification. Percent viability is shown relative to control cells treated with 0.1% DMSO. (FIG. 3C) Cytotoxicity of 5-aza-dC in MEFs from ifnar^{+/+} or ifnar^{-/-} mice expressing endogenous WT p53 (transduced with a non-specific control shRNA construct) or shRNA against p53 (shp53). Cells were left untreated or treated with 10 μM 5-aza-dC for 120 hours before detection of viable cells by methylene blue staining. (FIG. 3D) Caspase-3,7 activity (cleavage of the fluorescent substrate AC-Devd-AMC) in MEFs from p53-WT, p53-null and ifnar^{-/-} mice and MEFs from ifnar^{-/-} mice expressing shRNA against p53 (shp53). Cells were treated with the indicated concentrations of 5-aza-dC for 48 hours before the caspase assay. (FIG. 3E) Western blot analysis of expression of the IFN-inducible protein p49 (and β-actin as a loading control) in SCCVII cells: intact (1), mock transfected (2), transfected with the GFP-expression construct plv-CMV-GFP (250 ng per well of a 6-well plate; used here and below for monitoring transfection efficiency) (3), transfected with plv-CMV-GFP as above together with 500 ng of RNA (rRNA-depleted fraction) from 5-aza-dC-treated p53-WT (lane 4) and p53-null MEFs (lane 5) and untreated p53-WT (lane 6) and p53-null MEFs (lane 7). Transfection of plv-CMV-GFP and double-stranded poly(I:C) RNA (1 μg, an efficient IFN-inducing agent) was used as a positive control (lane 8).

FIG. 4. Massive transcriptional upregulation of repetitive elements in p53-null MEFs treated with 5-aza-dC. The abundance of gamma-satellite (GSAT, FIG. 4A), B2 (FIG. 4B), B1 (FIG. 4C) and ncRNA (FIG. 4D) transcripts in RNA samples isolated from untreated p53-WT MEFs, 5-aza-dC-treated p53-WT MEFs, untreated p53-null MEFs, and 5-aza-dC-treated MEFs is shown on bar graphs relative to the abundance of β-actin mRNA (calculations are based on the results of total RNA sequencing) and on Northern blots. For Northern blots the positions of 18S and 28S rRNAs are indicated by arrowheads and ethidium bromide staining of the gel before transfer is shown in panel (FIG. 4A) as a common control for RNA loading and quality. (FIG. 4E) The overall abundance of RNA transcripts representing gamma-satellite DNA (“sat DNA”), SINEs (B1 and B2), ncRNAs, and ERVs (including IAPs) in the indicated cells (p53-null (KO) or p53-WT MEFs, untreated or treated with 5-aza-dC) is shown in “β-actin” units (y-axis). Pie diagrams show the proportion represented by each of the above-listed classes of RNAs in the pool of new transcripts induced by 5-aza-dC treatment in p53-null MEFs (top) and in the pool of transcripts present in untreated p53-null cells versus p53-untreated p53-WTcells (bottom) (FIG. 4F) Hypothetical scheme of formation of dsRNA by annealing of RNA-polymerase III-driven transcripts of B1 or B2 SINEs with B1 and B2 sequences present in antisense orientation in polymerase II-driven mRNAs. Introns within mRNA are spliced out before nuclear export so it is unlikely for a dsRNA to be formed by the annealing of pol III transcribed SINE sequence to SINE sequences within mRNA introns which is further recognized by pattern recognition receptors outside the nucleus, such as PKR.

FIG. 5. Structural features of major repetitive sequences that are transcriptionally repressed by p53. (FIG. 5A and (Fig. B). In FIG. 5, the B1 SINE sequence is SEQ ID NO:269; the B2 SINE sequence is SEQ ID NO:270; the Major gamma sequence is SEQ ID NO:271; the B1 sequence is SEQ ID NO:272; the B2 sequence is SEQ ID NO:273, and the GSAT sequence is SEQ ID NO:274. Comparison of putative p53-binding sites from B1, B2 and GSAT repeats with “activating” and “repressing” p53 binding consensus sequences (32). (FIG. 5C and FIG. 5D) p53 binding to its consensus sequence within a ³²P-labeled oligonucleotide was detected by electromobility shift assay (EMSA) (c, lane 1) and the specificity of the observed p53-probe complex was confirmed by supershift with the anti-p53 antibody Ab421 (lane 2). Unlabeled competitor oligonucleotides were added at 10:1, 20:1 and 40:1 molar excess over the labeled probe in lanes 3-5 (oligonucleotide containing the p53-binding consensus sequence, positive control), lanes 6-8 (an 84-bp oligonucleotide containing the first two B1-derived putative p53-binding sequences), and lanes 9-11 (a λpL promoter-derived oligonucleotides of similar length, negative control). Bands corresponding to p53-binding complexes were quantified by densitometry and normalized to free radio-labeled probe. (FIG. 5E) Relative frequency of deviations from consensus at specific nucleotides positions within putative p53-binding sites of B1 elements activated by 5-aza-dC in p53-null MEF. Mutation rates (calculated as p-values) determined for the indicated sets of nucleotides of the three putative p53-binding sites found in B1 consensus sequence. The directed minimal difference was calculated between p-values in the 5-aza-dC treated p53-KO sample versus all three samples. Analysis of each key position (highlighted in red) of the p53-binding sites within the SINE B1 revealed that the positive minimal differences (black bars) are indicative of lower mutation rates found in the treated p53-KO samples when compared to any other sample set comprising of other nucleotides from p53 recognition element (RE) (blue bars; shown only for one of four sets analyzed). (FIG. 5F) Comparison of the mutation rate in the key nucleotide positions with mutation rates in other nucleotides within putative p53-binding sites in all 4 series was performed by the voting method. For each series, the sum of squares of positive differences is chi-square distributed as well as chi-square distributed is the sum of squares of the negative differences. The first chi-square value indicates the overall voting of series' positions for lower rate of mutation in 5-aza-dC treated p53-KO. In opposite, the second chi-square value is voting for higher overall rate of mutations in the series. The ratio of these two values normalized by the corresponding degrees of freedom (numbers of positive and negative differences) is F-distributed. Shown as minus log-p-values of the F-test in key-position series (left most bar) and four control series (right four bars). The F-test p-values of lower mutation rate in the 5aza+p53ko sample across 5 series of positions are listed. Only one p-value (0.004 of the key-positions) is statistically significant.

FIG. 6. Detection of transcripts of repetitive elements in mouse tumor cell lines and spontaneous tumors. (FIG. 6A) Detection of mouse gamma-satellite (GSAT) sequences in total RNA from mouse tumor cell lines CT-26 (colon tumor), LLC-1 (Lewis-lung carcinoma), and SCC-VII (squamous cell carcinoma) left untreated or treated with 10 μM 5-aza-dC for 48 hours. Dot blotting was performed with 500 ng total RNA per dot and single-strand hybridization probes GSAT-F and GSAT-R. (FIG. 6B) Detection of IFNβ1, IRF-7, CXCL10 and β-actin (loading control) mRNA by RT-PCR in the cells described in (FIG. 6A). (FIG. 6C) Cytotoxicity of 5-aza-dC in LLC-1, CT26 and SCC-VII cells. Cells were treated with the indicated concentrations of 5-aza-dC for 5 days. Viability was determined by methylene blue staining and extraction, followed by spectrophotometric quantification. Percent viability is shown relative to control cells treated with 0.1% DMSO. (FIG. 6D) Top two panels: Gamma-satellite (GSAT) sequences were detected in total RNA from thymic lymphomas of p53-null mice (5 tumors (L1-L5) from 5 different mice assayed in duplicate) and in two normal thymuses (T4, T5) isolated from p53-null mice using dot blotting as described in (FIG. 6A). Bottom two panels: RT-PCR analysis of IFNβ1 and β-actin (loading control) mRNA expression. (FIG. 6E) Northern hybridization was used to detect SINE B1 sequences in total RNA from MMTV-her2/neu mammary tumors (8 tumors from 8 different mice). 18S and 28S rRNA levels detected by ethidium bromide staining confirmed equivalent RNA quality and loading for all tumors.

FIG. 7. Sex of the embryo does not influence sensitivity of MEF to 5-aza-dC. Embryos from uteri of several pregnant females were analyzed with similar results; results of a representative experiment are shown. MEF were genotyped to determine the sex of each individual embryo by RT-PCR (FIG. 7A). Both MEF isolated from male/female embryos were then tested to for toxicity to 5-aza-dC for 5 days. Cells were fixed and stained with methylene blue (FIG. 7B).

FIG. 8. Quiescent cells are resistant to cultivation in the presence of 5-aza-dC. Monolayer of primary mouse hepatocytes isolated from p53-wt and null liver of C57BL/6 mice were incubated with culture medium containing increasing concentration of 5-aza-dC for 5 days (time sufficient to reach complete killing of dividing p53-null hepatocytes), fixed and quantitated using methylene blue staining.

FIG. 9. 5-aza-dC treatment suppresses VSV infection in p53-null but not in p53-WT MEFs. Equally prepared cultures of p53-WT and p53-null MEFs, either left untreated or treated for 36 hours with 5-aza-dC, were infected with equivalent titers of VSV. Importantly, the virus was added at a time when there were no morphological or biochemical signs of toxicity of 5-aza-dC. The extent of viral infection was evaluated at 6 hours post-infection. Western blot analysis showed that 5-aza-dC treatment had no effect on the amount of viral P protein in p53-WT cells, but that it caused a dramatic reduction in the level of P protein in p53-null cells. Less efficient expression of viral proteins in p53-null versus p53-WT MEFs is a reproducible phenomenon which may reflect partial activation of IFN in p53-null cells prior to treatment with 5-aza-dC.

FIG. 10. Induction of transcription of intracisternal A-particles (IAPs) by 5-aza-dC. p53-WT and p53-null MEF were incubated 48 hrs in culture medium containing 10 μM of 5-aza-dC. Total RNA was isolated from these and control (untreated) MEF and the presence of IAP-specific transcripts was determined using Northern hybridization with 32P-labeled cDNA probes.

FIG. 11. Competition of 84-bp oligonucleotide containing first two B1-derived putative p53-binding sequence for p53 binding to its consensus sequence within ³²P-labeled oligonucleotide in electromobility shift assay. Unlabeled oligonucleotide of similar length with four key nucleotide positions within putative p53-binding sites replaced was used as negative control (each added in 10:1 and 15:1 molar access over labeled oligonucleotides). Specificity of observed band to p53 was proven by using anti-p53 antibodies Ab421 that caused band supershift. Densitometry-based quantitation of competition assay is shown in a right panel (density of bands corresponding to p53 were normalized to the signals from free radio-labeled probes).

### DETAILED DESCRIPTION

Mammalian genomes contain an abundance of non-coding DNA sequences as well as multiple classes of interspersed repetitive elements such as DNA transposons and retrotransposons. They vary in length and copy number, are evolutionarily younger than structural genes, and are generally considered “genomic junk” with uncertain physiological roles (1). Among these, the most abundant are short interspersed nuclear elements (SINEs) that originate from reverse transcription products of certain RNA pol III-driven transcripts (e.g., 7SL RNA and tRNAs) (2) and have been shown to retain some ability to retrotranspose (3,4). In addition, the mammalian genome also contains tandemly organized repeats known as satellite DNAs (5). These highly reiterated sequences are the main component of the centromeric and pericentromeric heterochromatin and are considered important structural elements of the chromosome (6). Altogether, these classes of non-coding DNA sequence elements occupy more than 50% of the human and mouse genomes (1).

Analysis of the phylogeny of SINEs suggests that they accumulated through multiple “explosions,” or bursts of amplification, which started about 65 million years ago and were intermitted by periods of dormancy (7,8). The explosive nature of multiplication of SINEs and the fact that they all are located predominantly in intergenic areas, pseudogenes and non-coding regions of genes suggest that their bursts of amplification were genetic catastrophes associated with massive loss of those genomes in which SINEs inserted within essential genes (2,9,10) and presumably contributed to the diversity of mammalian species. In fact, integration of new SINE copies into coding or regulatory sequences has the potential to disturb gene expression (11). Without intending to be constrained by any particular theory, that maintenance of the genetic stability of mammalian cells and organisms depends on their ability to prevent expression and amplification of SINEs. In fact, SINEs are not normally transcribed as distinct autonomous elements, but rather as integral parts of non-coding regions of larger protein-coding transcripts (2). The vast majority of SINEs are located in heavily methylated regions of DNA, which is believed to be one of the mechanisms of their epigenetic silencing (12,13).

However, under certain circumstances both retroelements and some classes of satellite DNA can be transcribed. The events triggering such activation are not well understood, but have been shown to include a variety of stresses such as heat shock (14, 15), viral infections (16), various DNA-damaging treatments (17), and inhibition of translation (14). In addition, transcription of both retroelements and satellite DNA was observed in tumors in a variety of models of mouse and human origin (18). Both the mechanisms underlying transcriptional activation of normally “silent” non-coding elements and repeats in stressed and transformed cells and the physiological consequences of this activation has heretofore remained poorly understood.

In the present disclosure, we show that tumor suppressor p53, known as a positive and negative regulator of transcription, plays a significant role, along with DNA methylation, in epigenetic silencing of classes of retroelements and satellite DNA in mice. Failure of this p53 function enables transcription of several classes of normally silent genomic DNA repeats, resulting in the induction of an interferon (IFN) response, which can be suicidal. We refer to this process as “TRAIN” which represents the discovery that “Transcription of Repeats activates INterferon”. In one embodiment which is described more fully below, we performed a high-throughput screen which revealed striking transcriptional upregulation of numerous genetic elements belonging to several classes of normally transcriptionally silent repeats and non-coding (nc) RNAs in p53-null, but not in p53-WT cells, treated with 5-aza-dC. Total abundance of new transcripts can be estimated as X % of total cellular RNA, which in various embodiments is used to detect and/or quantitate TRAIN. Transcripts which are involved with TRAIN are described in the entries in Tables 1, 2 and 3, and in SEQ ID NO:276 through SEQ ID NO:351, inclusive, and including all SEQ ID NO:s there between. The invention includes testing and/or quantitating any one, and any combination and/or sub-combination of transcripts in Tables 1, 2 and 3 SEQ ID NO:276 through SEQ ID NO:351. The invention also includes testing only any one or any such combination or sub-combination of transcripts, and thus in embodiments any transcript(s) can be excluded from being detected and/or quantitated.

The present disclosure is based in part on our observations with respect to the loss or inactivation of tumor suppressor p53 as being associated with increased cell sensitivity to DNA demethylation. These discoveries indicate that p53 and IFN cooperate through regulation of transcription and cell death, respectively, to prevent accumulation of cells with “unleashed” repeats. The present disclosure thus reveals a novel function of p53 that, in cooperation with DNA methylation, keeps large families of interspersed and tandem repeats transcriptionally dormant. In this regard, we demonstrate that treatment of p53-deficient, but not p53-WT, cells with the DNA demethylating agent 5-aza-dC was shown to cause transcriptional derepression of several classes of normally silent interspersed repeats (e.g., SINEs such as B1 and B2 repeats), tandem DNA satellites (e.g., GSAT), and non-coding RNAs (ncRNAs). Together, these elements represent a significant proportion of the mouse genome (>10%) and, when transcribed, give rise to new RNA species comparable in their abundance to the bulk of cellular mRNA.

Transcriptional derepression of repeats resulting from a combined lack of p53 function and DNA methylation was accompanied by induction of the classical type I IFN signaling pathway, which is driven in MEFs is driven by IFN-131 and leads to apoptotic cell death. The critical role played by the IFN response in death of MEFs under conditions of TRAIN was demonstrated using cells deficient in IFN receptor expression. While the precise mechanism underlying this toxicity remains to be determined, the present disclosure is consistent with evidence indicating that activation of a strong IFN response by dsRNA can result in apoptosis mediated either by induction of the pro-apoptotic Protein Kinase R (PKR) and 2′-5′-Oligoadenylate Synthetase (OAS)/RNase L pathways or activation of TRAIL/FAS death receptors (24).

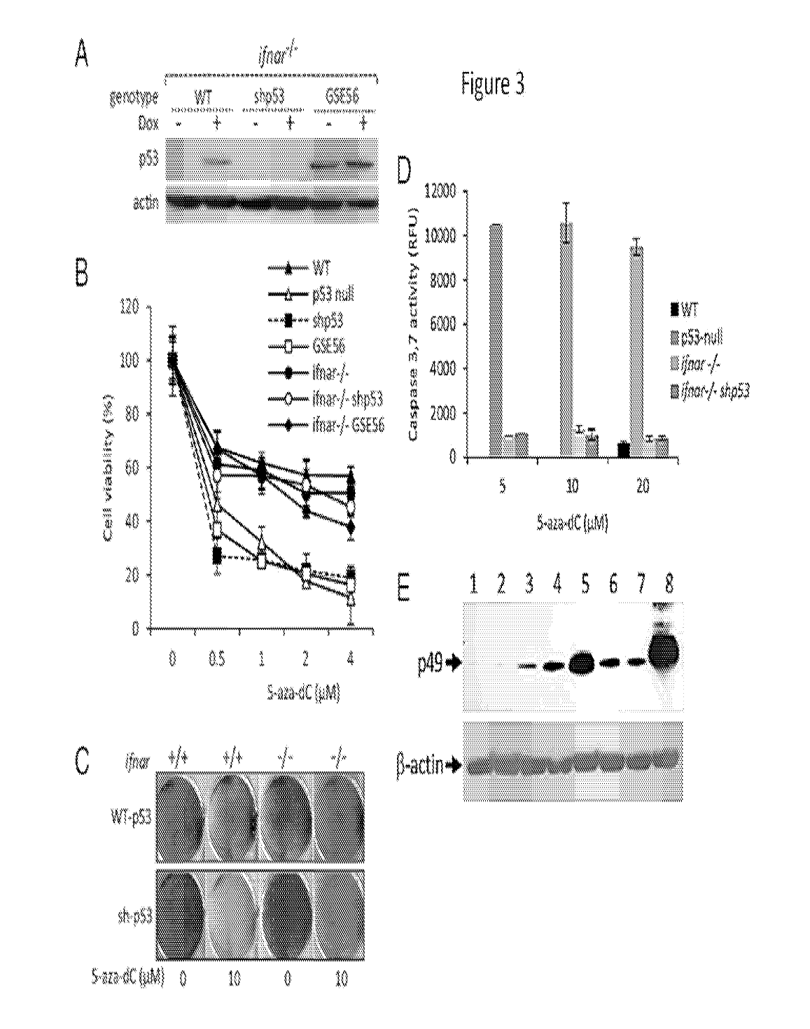

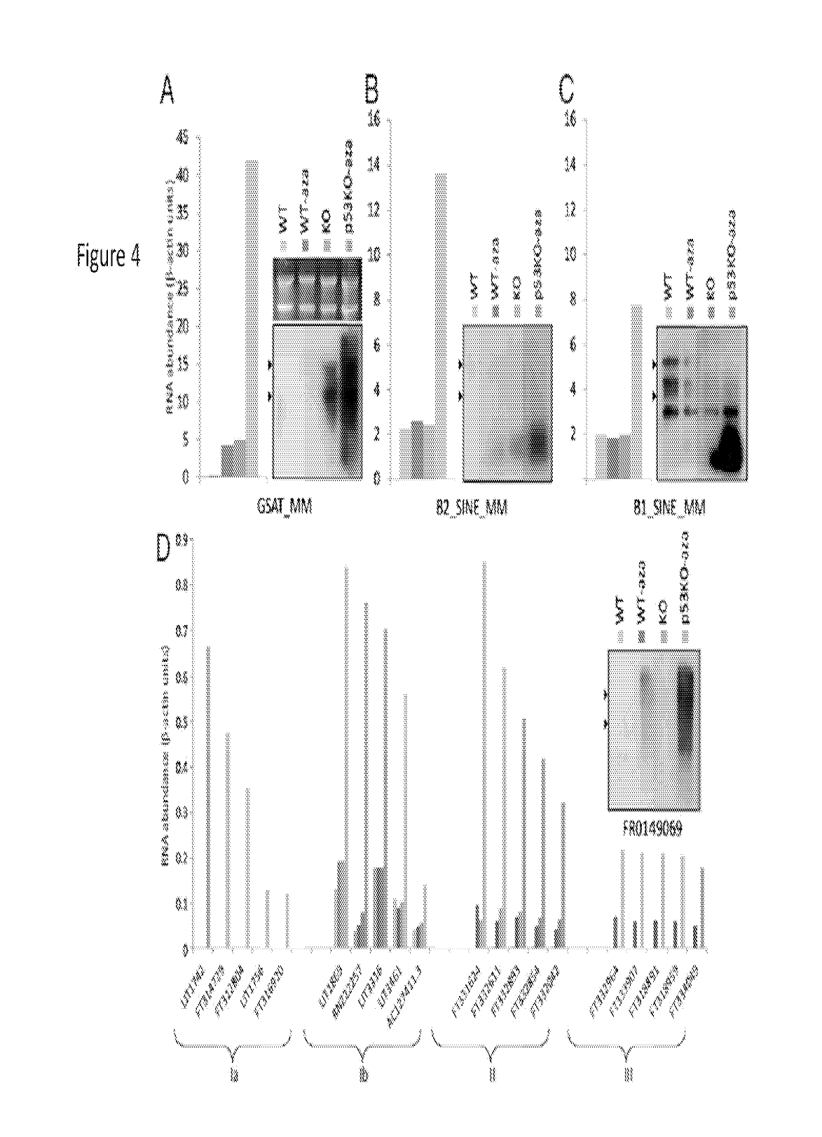

In mammalian cells, IFN signaling is commonly triggered as an antiviral defense mechanism in response to dsRNA produced during viral replication (24). Our results indicate that RNA species produced following transcriptional derepression of endogenous repeats in the absence of p53 and methylation can also trigger the IFN response. Transcripts of repetitive elements, retrotransposons and satellite DNA sequences can form dsRNA due to their intense secondary structure and the presence of complementary transcripts in the cell. For SINEs, such complementary sequences exist in non-coding regions of mRNAs that were transcribed through SINEs integrated in their antisense orientation (see FIG. 4F, described in the Examples). In the case of GSAT, both strands are transcribed, thereby producing complementary RNA strands (30). We have shown that total RNA from 2-aza-dC-treated p53-null cells has stronger IFN-inducing capacity than from untreated p53-null cells as well as from both treated and untreated p53-WT cells (see FIG. 3E in the Examples). We have determined that the scale of expression of “new” transcripts that appear in p53−/− cells treated with 5-aza-dC is so strong and their copy number in the genome is so high that we believe it would preclude use of gene knockout or knockdown technique to directly assess this. Nevertheless, since transcripts of SINEs and GSAT, both of which are capable of forming dsRNAs, comprise the major proportion of new RNA synthesized in p53-null cells following DNA hypomethylation, it is highly probably that these RNA species are responsible for IFN induction.

In general, and without intending to be bound by any particular theory, the results described in this disclosure support a model in which epigenetic silencing of repeats (believed to be an essential condition for genomic stability and viability of currently existing species) is controlled by three factors: (i) p53-mediated transcriptional silencing, (ii) DNA methylation-mediated suppression of transcription, and (iii) a suicidal IFN response which eliminates cells that escape the first two lines of control. Again without intending to be constrained by theory, this is believed to present a new role for p53 as a “guardian of repeats”, which is likely a component of its function as a “guardian of the genome” (43). For example, for at least interspersed repeats (products of reverse transcription), transcription is believed to be an essential prerequisite for their amplification and subsequent possible insertional mutagenesis. Given the abundance of repeats in mammalian genomes and their potential for producing large amounts of transcripts (which in the presence of reverse transcriptase may be converted into insertional mutagens), it is reasonable to state that silencing of repeats is likely an evolutionarily important function of p53 that may have been critical for survival of predecessors of current species during times of active repeat amplification. The present disclosure is also believed to reveal a new function for the IFN response in addition to its role in antiviral innate immunity: maintenance of genomic stability through elimination of cells that have lost epigenetic silencing of interspersed and tandem repeats. While results presented in this disclosure were obtained in mice, there are numerous indications that the process we have discovered and disclosed herein for the first time as TRAIN may be universal among mammals. For example, aberrant overexpression of satellite repeats was reported in pancreatic and other epithelial human cancers (18). In addition, hypomethylation and activation of polymerase III-driven Alu (the only major SINE in the human genome) transcription was found in human tumors (44, 45). It is considered that TRAIN is likely to be the explanation for the previously described phenomenon of inhibition of growth of pancreatic tumor cells by 5-aza-dC accompanied with the induction of IFN response signaling (46).

Thus, in view of the foregoing, it will be apparent to those skilled in art that TRAIN may explain a series of previously unconnected, but well-documented properties of tumor cells, including transcription of repeats, deregulation of IFN function and increased sensitivity to lytic viruses

Based at least in part on the foregoing, the present invention provides, in one embodiment, a method for detection of cancer or a pre-cancerous condition in a mammal comprising: detecting cells in the mammal, or in a sample obtained or derived from the mammal, RNA transcripts transcribed from repeats of polynucleotide sequences, the transcription of which is normally repressed by a combination of functional p53 and DNA methylation.

In one embodiment, the detecting is performed by detection of the RNA transcripts in a sample of blood obtained or derived from the individual. Samples analyzed in the method of the invention can be any sample, including tissues, such as but not limited to biopsies, hair, biological fluids, and any other biological sample that does or would be expected to contain polynucleotides and/or cells. The biological sample may be subjected to a processing step before performing the various steps of methods provided by the invention.

In non-limiting embodiments, for the detection of any RNA transcripts disclosed herein using any embodiment or aspect of the present disclosure, the method includes identifying and/or quantitating an amount of one or more the transcripts identified herein as being involved with TRAIN. In embodiments, the amount of the transcripts can be compared to any suitable reference (i.e., a control), examples of which include but are not limited to samples obtained from individuals or cell lines which have either normal or non-functional p53, or are null-p53 controls, or are matched controls, and/or experimentally designed controls such as known input RNA used to normalize experimental data for qualitative or quantitative determination of the amounts of transcripts listed in Tables 1, 2 and 3. The reference level may also be depicted graphically as an area on a graph. In certain embodiments, determining an amount of one or a plurality of transcripts in a sample above a threshold value is indicative of TRAIN in the sample, or in the tumor or in the individual from whom the sample was obtained. Likewise, determining an amount of one or a plurality of transcripts in a sample equal to or below a threshold value is an indication that the sample did not have TRAIN. Illustrative and non-limiting threshold values are shown in Tables 1, 2 and 3. In certain embodiments, the threshold difference relative to a reference is at least a one, two, three, four, or a fivefold difference. In one embodiment, an increase of the amount of at least one transcript described in the Tables and SEQ ID NO:s herein in a sample relative to a reference by at least fivefold is an indication that the sample exhibited TRAIN. As with qualitative or quantitative determination of the amounts of transcripts by comparison with a reference, the disclosure also includes qualitative or quantitative measurement of interferon by comparison with any suitable reference.

In an embodiment, the invention provides a method for detection of cancer or a pre-cancerous condition in a mammal by detecting cells in the individual, or in a sample obtained or derived from the individual, wherein the cells are characterized by a constitutively active type I interferon response. In certain embodiments, this can be performed by detecting RNA or proteins, or a combination thereof, wherein the RNA and/or the protein is encoded by an interferon-responsive gene.

In another embodiment, the invention provides a method for predicting tumor sensitivity to 5-aza-dC treatment by determining the absence of indicia of TRAIN, wherein the indicia of TRAIN comprise: i) ncRNA species and/or RNA transcripts transcribed from polynucleotide sequence repeats, examples of which are presented in the Tables, and particularly Table 3, the transcription of which is normally suppressed by functional p53, or ii) constitutive activation of an interferon type I response; or iii) a combination of i) and ii).

In another embodiment, provided is a method for prophylaxis and/or treatment of cancer by selective elimination of cells with inactivated p53, the method comprising administering to an individual in need of the prophylaxis and/or therapy a composition comprising an effective amount of one or more agents which induce DNA demethylation. The one or more agents can be provided as a pharmaceutical preparation, which can contain a pharmaceutically acceptable carrier. The one or more agents can be delivered to an individual using any suitable method, including but not necessarily limited to oral, parenteral, subcutaneous, intraperitoneal, intrapulmonary, intranasal and intracranial injections. Parenteral infusions include intramuscular, intravenous, intraarterial, intraperitoneal, and subcutaneous administration. In certain embodiments, the method of the invention can be performed prior to, concurrently, or subsequent to conventional anti-cancer therapies, including but not limited to chemotherapies, surgical interventions, and radiation therapy. It will be recognized by those of skill in the art that the form and character of the particular dosing regimen for any composition administered to an individual according to the method of the invention will be dictated at least in part by the route of administration and other well-known variables, taking into account such factors as the size, gender, health and age of the individual to be treated. Cells/tumors with inactivated p53, and inactivated p53 itself, can be identified by the skilled artisan in view of the present disclosure.

In another embodiment, the invention provides a method of selection and/or identification of agents which are capable of DNA demethylation in cells with inactivated p53. This method comprises contacting cells with inactivated p53 with one or more test agents, and determining whether or not the test agents induce activation of transcription of repeats and/or ncRNA species which are normally suppressed by functional p53.

In another embodiment, the invention provides a method of classification of a tumor, the method comprising determining whether or not the tumor exhibits indicia of TRAIN, wherein the indicia of TRAIN comprise: i) ncRNA species and/or RNA transcripts transcribed from polynucleotide sequence repeats, the transcription of which is normally suppressed by functional p53, or: ii) constitutive activation of an interferon type I response; or iii) a combination of i) and ii).

In yet another embodiment, the invention provides a method of determination of functional status of p53 in a cell or tumor, the method comprising determining whether or not the cell or the tumor exhibits the aforementioned indicia of TRAIN. In an embodiment, the determination of functional status of p53 in a cell or tumor, or in any other sample, comprises contacting the cell and/or tumor with a demethylation agent and determining one or a plurality of the transcripts described herein, wherein a change in the amount of the one or more of the transcripts relative to a reference indicates that the p53 in the cell or tumor is non-functional.

In another embodiment, provided is a method of determination of tumor sensitivity to treatment with oncolytic virus therapy by detection of indicia of TRAIN, wherein the presence of indicia of TRAIN is indicative that the tumor is likely to be sensitive to oncolytic virus therapy. In embodiments, the method further comprises administering oncolytic virus therapy to the individual.

In another embodiment, the invention provides a method of determination of tumor sensitivity to interferon therapy by detection of indicia of TRAIN in the tumor, wherein the presence of the indicia of TRAIN is indicative that the tumor is likely to be sensitive to interferon therapy. In an embodiment, this method further comprises administering interferon therapy to the individual.

In another embodiment, the invention provides a method of detection of TRAIN comprising using in situ hybridization techniques with oligonucleotides specific for transcripts and/or ncRNA species characteristic of TRAIN. Any suitable in situ hybridization techniques can be used and will be recognized by those skilled in the art. In certain embodiment, for all aspects of the invention, the transcripts and/or ncRNA species are selected from the sequences described in Table 1 and/or Table 2 and/r Table 3, which are each designated by GenBank accession numbers, and for which each sequence associated with each entry is incorporated herein by reference as of the date of filing of this application. Table 1 describes genes upregulated five or more times in p53-WT MEF treated with 5-aza-dC compared to untreated p53-WT MEF (average signals from microarray hybridization results). Table 2 describes a list of genes upregulated five or more times in p53-null MEF treated with 5-aza-dC compared to untreated p53-null MEF (average signals from microarray hybridization results). Table 3 describes a list of ncRNAs in p53-WT and p53-null MEFs treated and untreated with 5-aza-dC based on RNA-seq analysis.

TABLE 1

| | | | p53 WT | | p53-null |
|---|---|---|---|---|---|
| Target ID | Accession No. | p53 WT | 5-aza-dC | p53-null | 5-aza-dC |
| IAP (SEQ ID NO: 1) | NM_010490.2 | 1114.7 | 14029.55 | 15715.6 | 18332.9 |
| LOC100043821 (SEQ ID | NC_00080.5 | 145.5 | 4021.55 | 4034.6 | 8040.8 |
| NO: 2) | | | | | |
| MRGPRF (SEQ ID NO: 3) | NM_145379.2 | 292.7 | 1465.2 | 1997 | 1347.9 |
| PSG23 (SEQ ID NO: 4) | NM_020261 | 9.7 | 1232.05 | 667.2 | 5114.7 |
| ZCCHC5 (SEQ ID NO: 5) | NM_199468.1 | 187.8 | 1193.45 | 1643.4 | 677.4 |
| AQP1 (SEQ ID NO: 6) | NM_007472.2 | 100.4 | 1146.25 | 1617.7 | 3771.25 |
| LOC280487 (SEQ ID NO: 7) | | 20.1 | 1052.3 | 1160.6 | 3292.7 |
| NRN1 (SEQ ID NO: 8) | NM_153529.1 | 147.2 | 998.85 | 1354.3 | 1648.6 |
| SCX (SEQ ID NO: 9) | NM_198885 3 | 150.1 | 966.8 | 1099.4 | 722.55 |
| ENO3 (SEQ ID NO: 10) | NM_007933.2 | 182.5 | 936.25 | 1076 | 1821.55 |
| LOC386135 (SEQ ID NO: 11) | | 6.8 | 810.35 | 918.2 | 2483.6 |
| RSPO2 (SEQ ID NO: 12) | NM_172815.1 | 57.7 | 788.2 | 1118.3 | 646.7 |
| ABI3BP (SEQ ID NO: 13) | NM_178790.3 | 132.5 | 695.35 | 947.9 | 622.75 |
| CXCL15 (SEQ ID NO: 14) | NM_011339.2 | 9.2 | 675.55 | 1031 | 653.7 |
| COMP (SEQ ID NO: 15) | NM_016685.2 | 7 | 478.55 | 765.8 | 737.8 |
| MND1 (SEQ ID NO: 16) | NM_029797.3 | 69 | 437.75 | 455.9 | 1365.5 |
| SLPI (SEQ ID NO: 17) | NM_011414.3 | 75.9 | 396.9 | 345 | 882.55 |
| PENK1 (SEQ ID NO: 18) | NM_001002927.2 | 4 | 382.65 | 503.5 | 559.4 |
| HOXD13 (SEQ ID NO: 19) | NM_008275.2 | 22.2 | 348.95 | 467.8 | 439.95 |
| LOC100047226 (SEQ ID | | 63.7 | 325.4 | 435.1 | 1045.5 |
| NO: 20) | | | | | |
| RHOX5 (SEQ ID NO: 21) | NM_008818.2 | 30.2 | 323.4 | 173.4 | 661.2 |
| E430033B07RIK (SEQ ID | | 29.6 | 312.95 | 132.1 | 1006.5 |
| NO: 22) | | | | | |
| AQP5 (SEQ ID NO: 23) | NM_009701.4 | 40 | 302.95 | 417.3 | 588.35 |
| BCAT2 (SEQ ID NO: 24) | NM_001243052.1 | 48.1 | 297.55 | 323.3 | 161.2 |
| SLC29A1 (SEQ ID NO: 25) | NM_022880.1 | 47.7 | 246.45 | 300.8 | 521.85 |
| LTBP4 (SEQ ID NO: 26) | NM_175641.1 | 36.4 | 244.5 | 264.2 | 326.95 |
| FMOD (SEQ ID NO: 27) | NM_021355.2 | 37 | 233.25 | 319.4 | 176.75 |
| UNC5C (SEQ ID NO: 28) | NM_152823.3 | 23.1 | 229.2 | 303.6 | 294.6 |
| ANPEP (SEQ ID NO: 29) | NM_008486.2 | 18 | 213.55 | 202.2 | 107.75 |
| MEOX1 (SEQ ID NO: 30) | NM_010791.3 | 9.2 | 211.95 | 318.2 | 122.45 |
| TBX4 (SEQ ID NO: 31) | NM_011536.2 | 30.4 | 199.7 | 262.5 | 239.55 |
| TEX19.1 (SEQ ID NO: 32) | NM_028602.2 | 11.6 | 185.55 | 168.1 | 654.9 |
| HOXA11S (SEQ ID NO: 33) | | 21.9 | 178.85 | 206.7 | 183.35 |
| MME (SEQ ID NO: 34) | NM_008604.2 | 29.9 | 171.8 | 205.2 | 243.35 |
| C1QTNF5 (SEQ ID NO: 35) | | 31.1 | 171.1 | 202.2 | 156.4 |
| MYL6B (SEQ ID NO: 36) | NM_172259.1 | 19.9 | 168.35 | 157 | 94.05 |
| HOPX (SEQ ID NO: 37) | NM_010629 | 18.3 | 158.45 | 191.1 | 407.95 |
| CDSN (SEQ ID NO: 38) | NM_001008424.2 | 18.9 | 157.85 | 243.5 | 289.2 |
| GJB3 (SEQ ID NO: 39) | NM_008126.1 | 27 | 146.95 | 128.4 | 332.05 |
| RARRES1 (SEQ ID NO: 40) | NM_001164763.1 | 26.4 | 142.1 | 161.6 | 407.1 |
| DPEP1 (SEQ ID NO: 41) | NM_007876.1 | 26 | 132.95 | 102.5 | 409.75 |
| SLC25A45 (SEQ ID NO: 42) | NM_134154.3 | 23.4 | 121.7 | 90 | 174.95 |
| HTRA3 (SEQ ID NO: 43) | NM_030127.2 | 19.4 | 119.15 | 115 | 105.85 |
| LTB4R1 (SEQ ID NO: 44) | NM_008519.2 | 15.6 | 115.2 | 69.1 | 11.9 |
| FAM132B (SEQ ID NO: 45) | NM_173395.2 | 22.1 | 113.35 | 127.1 | 162.35 |
| A030010B05RIK (SEQ ID | NM_030100 | 18.7 | 111.3 | 119.1 | 110.6 |
| NO: 46) | | | | | |
| DNM1 (SEQ ID NO: 47) | NM_010065.2 | 12.2 | 108.85 | 149 | 53.5 |
| LOC100046616 (SEQ ID | | 14.8 | 104.6 | 131.8 | 202.65 |
| NO: 48) | | | | | |
| CDA (SEQ ID NO: 49) | NM_028176.1 | 9 | 99.15 | 101.9 | 185.25 |
| EPN1 (SEQ ID NO: 50) | NM_001252454.1 | 17.6 | 98.55 | 91.5 | 51 |
| BC018371 (SEQ ID NO: 51) | | 4.6 | 98.5 | 103 | 49.15 |
| GSTP2 (SEQ ID NO: 52) | NM_181796.2 | 9.1 | 98.4 | 91.9 | 419.85 |

TABLE 2

| | | | p53-WT | | p53-null |
|---|---|---|---|---|---|
| Target ID | Accession No. | p53-WT | 5-aza-dC | p53-null | 5-aza-dC |
| IFNB1 (SEQ ID NO: 53) | NM_010510.1 | 5 | 1.3 | 0.2 | 407.85 |
| CXCL10 (SEQ ID NO: 54) | NM_021274.1 | 74.6 | 125.3 | 112.5 | 7766.4 |
| LOC432555 (SEQ ID NO: 55) | NM_001024230.2 | 0.8 | 12.65 | 11.3 | 740.95 |
| CXCL9 (SEQ ID NO: 56) | NM_008599.4 | 9.4 | 40.8 | 31.6 | 2062.95 |
| 4930438A08RIK (SEQ ID | XM_001477470.2 | 0.6 | 13.4 | 7.9 | 477.3 |
| NO: 57) | | | | | |
| EG433016 (SEQ ID NO: 58) | NM_001082547.1 | 13.2 | 5 | 2.8 | 135.45 |
| MX1 (SEQ ID NO: 59) | NM_010846.1 | 8.1 | 18.4 | 18.9 | 850.7 |
| IRGB10 (SEQ ID NO: 60) | NM_001135115.1 | 20.9 | 23.5 | 22.6 | 979.95 |
| KRT16 (SEQ ID NO: 61) | NM_008470.1 | 2.3 | 13.4 | 2.4 | 98.8 |
| POU3F1 (SEQ ID NO: 62) | NM_011141.2 | 18.4 | 3.5 | 2.7 | 110.65 |
| 9930111J21RIK (SEQ ID | NM_001114679.1 | 12.9 | 18.55 | 22 | 753.85 |
| NO:63) | | | | | |
| 2010002M12RIK (SEQ ID | NM_053217.2 | 2.1 | 3.45 | 7.6 | 248.9 |
| NO: 64) | | | | | |
| CCL4 (SEQ ID NO: 65) | NM_013652.2 | 260.9 | 45.2 | 29.1 | 573.35 |
| MX2 (SEQ ID NO: 66) | NM_013606 | 166.3 | 301.85 | 299 | 5885.7 |
| OASL1 (SEQ ID NO: 67) | NM_145209.3 | 59.1 | 100.55 | 80.6 | 1551.45 |
| CD274 (SEQ ID NO: 68) | NM_021893.3 | 117.2 | 124.2 | 91.5 | 1720.9 |
| RSAD2 (SEQ ID NO: 69) | NM_021384.3 | 405.6 | 493.3 | 509.9 | 9355.1 |
| P2RX7 (SEQ ID NO: 70) | NM_001038839.1 | 2.4 | 19.6 | 11.4 | 206.25 |
| IFI47 (SEQ ID NO: 71) | NM_008330.1 | 89.3 | 74.8 | 62.3 | 1123.75 |
| CCL5 (SEQ ID NO: 72) | NM_013653.2 | 64.7 | 106.5 | 89.6 | 1536.15 |
| 8030474K03RIK (SEQ ID | XM_001000772.2 | 1.1 | 15.3 | 6.1 | 102.55 |
| NO: 73) | | | | | |
| 3110043J09RIK (SEQ ID | NM_001164627.1 | 2.4 | 13.45 | 8 | 132 |
| NO:74) | | | | | |
| PRF1 (SEQ ID NO: 75) | NM_011073.2 | 12 | 14.9 | 7.1 | 113.55 |
| LOC100040016 (SEQ ID | NM_001085348.1 | 4.6 | 18.1 | 8.6 | 129.25 |
| NO: 76) | | | | | |
| OTTMUSG00000016644 | NM_001101605.1 | 23.3 | 32.55 | 28.2 | 418.25 |
| (SEQ ID NO: 77) | | | | | |
| LOC623121 (SEQ ID NO: 78) | NM_001177349.1 | 35.7 | 48.65 | 46.6 | 675.85 |
| RHBDL2 (SEQ ID NO: 79) | NM_183163.2 | 56.6 | 46.45 | 14.5 | 204.55 |
| AA467197 (SEQ ID NO: 80) | NM_001004174.1 | 32.6 | 119.25 | 128.9 | 1789.15 |
| ISG20 (SEQ ID NO: 81) | NM_001113527.1 | 14.2 | 42.9 | 46.1 | 628.2 |
| LOC676133 (SEQ ID NO: 82) | XR_004881.1 | 4.1 | 26.1 | 12.5 | 164.2 |
| CCRL2 (SEQ ID NO: 83) | NM_017466.4 | 16.5 | 26.25 | 19.5 | 235 |
| TYKI (SEQ ID NO: 84) | NM_020557.3 | 234.3 | 198.65 | 212.7 | 2541.75 |
| BC030476 (SEQ ID NO: 85) | NM_173421.1 | 39 | 31.05 | 15 | 178.35 |
| IFI205 (SEQ ID NO: 86) | NM_172648.3 | 39.9 | 47.45 | 45.1 | 528.2 |
| DPEP3 (SEQ ID NO: 87) | NM_027960.2 | 5.2 | 20.85 | 9.1 | 106.45 |
| CASP1 (SEQ ID NO: 88) | NM_009807.2 | 34.9 | 87.35 | 53.1 | 611.25 |
| GBP6 (SEQ ID NO: 89) | NM_145545.2 | 63 | 61.9 | 43.5 | 497.65 |
| A530057A03RIK (SEQ ID | NM_001081089.1 | 1.2 | 24 | 13.9 | 156.75 |
| NO: 90) | | | | | |
| D14ERTD668E (SEQ ID | NM_199015.2 | 184.1 | 361.85 | 365 | 3893.5 |
| NO: 91) | | | | | |
| LOC675594 (SEQ ID NO: 92) | XR_005116 | 13.2 | 29.05 | 14.6 | 153.05 |
| IL15 (SEQ ID NO: 93) | NM_008357.1 | 24.4 | 32.95 | 23.9 | 247.3 |
| SCT (SEQ ID NO: 94) | NM_011328.2 | 14 | 38.65 | 21.2 | 217.3 |
| TAF7L (SEQ ID NO: 95) | NM_028958.3 | 3.3 | 147.6 | 99.5 | 1011.15 |
| 4930599N23RIK (SEQ ID | NR_045813.1 | 11.8 | 29.65 | 21.3 | 203.65 |
| NO: 96) | | | | | |
| HAMP2 (SEQ ID NO: 97) | NM_183257.3 | 5.3 | 37.6 | 27.2 | 259.25 |
| SECTM1A (SEQ ID NO: 98) | NM_145373.2 | 8.1 | 17.75 | 11.4 | 107.05 |
| IRF7 (SEQ ID NO: 99) | NM_016850.2 | 164.1 | 260.8 | 224.4 | 2098.6 |
| C4A (SEQ ID NO: 100) | NM_011413.2 | 11.8 | 22.25 | 18.8 | 173.15 |
| NRAP (SEQ ID NO: 101) | NM_008733.3 | 13.4 | 14.5 | 11.5 | 101.2 |
| CASP4 (SEQ ID NO: 102) | NM_007609.1 | 44 | 66.4 | 60.2 | 527.45 |
| PSMB9 (SEQ ID NO: 103) | NM_013585.2 | 151.8 | 172.8 | 139.2 | 1208.7 |
| HAP1 (SEQ ID NO: 104) | NM_010404.3 | 63.6 | 25.6 | 22.7 | 190.55 |
| TRIM69 (SEQ ID NO: 105) | NM_080510.2 | 31.6 | 28.5 | 15.5 | 128.2 |
| CNKSR1 (SEQ ID NO: 106) | NM_001081047.1 | 5.6 | 12.45 | 15.6 | 128.15 |
| SASS6 (SEQ ID NO: 107) | NM_028349.2 | 89.8 | 51.8 | 31.4 | 255.8 |
| OAS2 (SEQ ID NO: 108) | NM_145227.3 | 102.7 | 140.95 | 101.4 | 814.4 |
| ARC (SEQ ID NO: 109) | NM_018790.2 | 30.8 | 86.4 | 90 | 711.05 |
| TUBA3B (SEQ ID NO: 110) | NM_009449.3 | 43.8 | 170.65 | 95.9 | 741.75 |
| WFDC12 (SEQ ID NO: 111) | NM_138684.2 | 3 | 20.55 | 23.1 | 178.45 |
| PSG23 (SEQ ID NO: 112) | NM_020261.4 | 9.7 | 1232.05 | 667.2 | 5114.7 |
| PIWIL2 (SEQ ID NO: 113) | NM_021308.1 | 14.3 | 31.4 | 27.1 | 206.25 |
| ZFP296 (SEQ ID NO: 114) | NM_022409.2 | 5.1 | 26.65 | 25.9 | 196.55 |
| LOC223672 (SEQ ID NO: 115) | NM_001162883.1 | 273.5 | 401.3 | 299.1 | 2257.4 |
| TNFAIP3 (SEQ ID NO: 116) | NM_009397.2 | 9.2 | 24.25 | 20.8 | 151.6 |
| PKD1L2 (SEQ ID NO: 117) | NM_029686.4 | 3.5 | 49.25 | 34.6 | 248.35 |
| ATF3 (SEQ ID NO: 118) | NM_007498.2 | 32.4 | 71.9 | 52.3 | 370.7 |
| ZBP1 (SEQ ID NO: 119) | NM_001139519.1 | 28.2 | 21.9 | 17 | 119.95 |
| HIST1H2AG (SEQ ID NO: 120) | NM_178186.2 | 30.4 | 46.4 | 20.1 | 141.6 |
| AI607873 (SEQ ID NO: 121) | NM_001204910.1 | 245.3 | 235.2 | 213.1 | 1497.35 |
| TUBA3A (SEQ ID NO: 122) | NM_009446.2 | 20.5 | 62.35 | 45.7 | 320.9 |
| EG240327 (SEQ ID NO: 123) | NM_001033767.3 | 256 | 146.35 | 132.5 | 909 |
| BC010462 (SEQ ID NO: 124) | BC010462 | 0.5 | 12.6 | 15.3 | 104.65 |
| IFIT2 (SEQ ID NO: 125) | NM_008332.3 | 293.3 | 411.25 | 506.1 | 3429.1 |
| GBP2 (SEQ ID NO: 126) | NM_010260.1 | 513.1 | 745.65 | 772.7 | 5218.75 |
| D630022O22RIK (SEQ ID | AK085407 | 34.4 | 23.9 | 37.4 | 248.85 |
| NO: 127) | | | | | |
| DAZL (SEQ ID NO: 128) | NM_010021.4 | 2.8 | 71.35 | 64.3 | 420 |
| LOC100047583 (SEQ ID | XM_001479138.1 | 1031 | 808.25 | 939.3 | 6057.85 |
| NO: 129) | | | | | |
| LOC383125 (SEQ ID NO: 130) | XM_356890.1 | 232.4 | 176.4 | 148 | 942.4 |
| LOC332788 (SEQ ID NO: 131) | XM_285750.4 | 75.2 | 53.25 | 50.6 | 321.35 |
| LOC268730 (SEQ ID NO: 132) | XM_193754.2 | 92.5 | 67.15 | 65.2 | 412.05 |
| LOC380839 (SEQ ID NO: 133) | XM_354746 | 0.1 | 40.15 | 29.6 | 186.9 |
| 0610037M15RIK (SEQ ID | NM_207648.1 | 21.8 | 46.3 | 30.5 | 190.4 |
| NO: 134) | | | | | |
| TRIM21 (SEQ ID NO: 135) | NM_009277.3 | 186 | 187.05 | 186.1 | 1150.9 |
| LOC229395 (SEQ ID NO: 136) | XM_130929.5 | 7.2 | 29.1 | 22.5 | 138.55 |
| SLC15A3 (SEQ ID NO: 137) | NM_023044.1 | 51.6 | 37.1 | 27.5 | 169.25 |
| DND1 (SEQ ID NO: 138) | NM_173383.2 | 11.1 | 29.7 | 16.6 | 99.95 |
| LOC386144 (SEQ ID NO: 139) | XM_359091.1 | 22.9 | 47.65 | 30.5 | 183.4 |
| LOC100047963 (SEQ ID | XM_001479238.1 | 280.8 | 252.05 | 258 | 1538.05 |
| NO: 140) | | | | | |
| ZXDA (SEQ ID NO: 141) | NR_003292.1 | 33.3 | 30.6 | 18.8 | 111.1 |
| APOL9B (SEQ ID NO: 142) | NM_001168660.1 | 73.7 | 245.5 | 229.1 | 1345 |
| LOC100041137 (SEQ ID | XR_032462.1 | 6 | 66.95 | 42.9 | 250.1 |
| NO:143) | | | | | |
| OAS1B (SEQ ID NO: 144) | NM_001083925.1 | 53.2 | 56.85 | 56.4 | 327.6 |
| CCL2 (SEQ ID NO: 145) | NM_011333.3 | 481.3 | 530.15 | 386.2 | 2231.1 |
| LOC385982 (SEQ ID | | 6.1 | 30.4 | 20.2 | 116.35 |
| NO: 146) | | | | | |
| H2-Q8 (SEQ ID NO: 147) | NM_023124.4 | 67.7 | 115.05 | 84.8 | 487.45 |
| LOC384348 (SEQ ID | | 80.7 | 68.9 | 63 | 361.95 |
| NO: 148) | | | | | |
| CYTIP (SEQ ID NO: 149) | NM_139200.4 | 8 | 19.25 | 20.4 | 117.2 |
| DCP2 (SEQ ID NO: 150) | NM_027490.1 | 99.3 | 113.6 | 104.1 | 590.8 |
| SAMHD1 (SEQ ID NO: 151) | NM_018851.2 | 139.4 | 144.55 | 139.5 | 786.45 |
| LOC667370 (SEQ ID | NM_001005858 | 873.6 | 1078.1 | 1493.2 | 8416.05 |
| NO: 152) | | | | | |
| IRF5(SEQ ID NO: 153) | NM_001252382.1 | 40.2 | 45.8 | 33.5 | 186.85 |
| APOL7A (SEQ ID NO: 154) | NM_001164640.1 | 23.6 | 99.75 | 96 | 524.9 |
| H2-Q6 (SEQ ID NO: 155) | NM_207648.1 | 13.6 | 20.25 | 18.9 | 102.4 |
| SLC11A1 (SEQ ID NO: 156) | NM_013612.1 | 57 | 62.25 | 33.7 | 181.65 |
| PARP14 (SEQ ID NO: 157) | NM_001039530.1 | 347.4 | 427.85 | 407.7 | 2172.45 |
| D11LGP2E (SEQ ID NO: 158) | NM_030150.2 | 78.2 | 126.05 | 122.1 | 649.9 |
| A1451557 (SEQ ID NO: 159) | NM_001033207.2 | 83 | 108.1 | 84.5 | 449.5 |
| DAXX (SEQ ID NO: 160) | NM_001199733.1 | 386.6 | 658.15 | 647.6 | 3422.95 |
| LOC239727 (SEQ ID | NM_181075.3 | 88.6 | 51.4 | 58.8 | 310.75 |
| NO: 161) | | | | | |
| 4930553M18RIK (SEQ ID | NM_027261.3 | 39.1 | 35 | 21.1 | 107.3 |
| NO: 162) | | | | | |
| DHX58 (SEQ ID NO: 163) | NM_030150.2 | 248 | 433.7 | 401.2 | 2035.75 |
| H2-M3 (SEQ ID NO: 164) | NM_013819.2 | 305.1 | 374.25 | 286.4 | 1451.1 |
| CDH15 (SEQ ID NO: 165) | NM_007662.2 | 31.2 | 27.5 | 23 | 116.25 |
| AIM1 (SEQ ID NO: 166) | NM_172393.2 | 18.8 | 43.65 | 37.7 | 189.85 |
| GBP3 (SEQ ID NO: 167) | NM_018734.2 | 862.7 | 1200.3 | 1332.6 | 6704.8 |
| TOR3A (SEQ ID NO: 168) | NM_023141 | 218.3 | 211.45 | 260.9 | 1312.6 |
| HIST1H2AN (SEQ ID | NM_178184.1 | 217.5 | 199.95 | 127.2 | 636.85 |
| NO: 169) | | | | | |
| CHKA (SEQ ID NO: 170) | NM_001025566.1 | 85.6 | 100.15 | 74.2 | 370.35 |

TABLE 3

| | | | p53 WT | | p53-null |
|---|---|---|---|---|---|
| Target ID | Accession No. | p53 WT | 5-aza-dC | p53-null | 5-aza-dC |
| FT312804 (SEQ ID NO: 171) | AK136884 | 1.2 | 1.2 | 1.0 | 1775.0 |
| FT314082 (SEQ ID NO: 172) | AK035603 | 14.2 | 131.0 | 17.2 | 466.0 |
| FT314729 (SEQ ID NO: 173) | AK036937 | 1.0 | 1.0 | 1.0 | 2379.0 |
| FT316398 (SEQ ID NO: 174) | AK138403 | 21.0 | 224.0 | 22.0 | 598.0 |
| FT316920 (SEQ ID NO: 175) | AK079647 | 1.0 | 1.0 | 1.0 | 618.0 |
| FT318403 (SEQ ID NO: 176) | AK041371 | 4.0 | 155.0 | 7.0 | 618.0 |
| FT318891 (SEQ ID NO: 177) | AK080354 | 9.2 | 316.0 | 11.0 | 1050.0 |
| FT318959 (SEQ ID NO: 178) | AK042254 | 13.7 | 305.0 | 15.2 | 1029.0 |
| FT319598 (SEQ ID NO: 179) | AK139172 | 8.2 | 243.0 | 10.0 | 865.0 |
| FT320180 (SEQ ID NO: 180) | AK043905 | 11.7 | 206.0 | 13.0 | 680.0 |
| FT320789 (SEQ ID NO: 181) | AK139771 | 18.5 | 243.0 | 21.0 | 756.0 |
| FT322471 (SEQ ID NO: 182) | AK140518 | 4.2 | 76.0 | 8.2 | 225.0 |
| FT32526 (SEQ ID NO: 183) | AK006721 | 993.0 | 1161.0 | 1.0 | 3349.0 |
| FT326003 (SEQ ID NO: 184) | AK050668 | 5.0 | 89.0 | 5.2 | 330.0 |
| FT326023 (SEQ ID NO: 185) | AK050666 | 5.2 | 93.0 | 8.0 | 251.0 |
| FT326254 (SEQ ID NO: 186) | AK083524 | 19.2 | 183.0 | 20.0 | 536.0 |
| FT331301 (SEQ ID NO: 187) | AK054341 | 19.2 | 211.0 | 20.2 | 657.0 |
| FT331624 (SEQ ID NO: 188) | AK153977 | 1.2 | 480.0 | 317.0 | 4249.0 |
| FT331672 (SEQ ID NO: 189) | AK088657 | 5.5 | 90.0 | 8.2 | 256.0 |
| FT331736 (SEQ ID NO: 190) | AK088947 | 4.7 | 83.0 | 7.5 | 263.0 |
| FT332042 (SEQ ID NO: 191) | AK154549 | 13.2 | 216.0 | 320.0 | 1614.0 |
| FT332332 (SEQ ID NO: 192) | AK089398 | 12.5 | 118.0 | 202.0 | 947.0 |
| FT332409 (SEQ ID NO: 193) | AK155639 | 5.2 | 69.0 | 8.2 | 208.0 |
| FT332481 (SEQ ID NO: 194) | AK089559 | 11.5 | 138.0 | 188.0 | 1096.0 |
| FT332611 (SEQ ID NO: 195) | AK171704 | 18.5 | 309.0 | 441.0 | 3087.0 |
| FT332624 (SEQ ID NO: 196) | AK171721 | 23.2 | 199.0 | 331.0 | 1604.0 |
| FT332735 (SEQ ID NO: 197) | AK089719 | 5.2 | 109.0 | 189.0 | 942.0 |
| FT332765 (SEQ ID NO: 198) | AK089731 | 7.2 | 82.0 | 144.0 | 597.0 |
| FT332798 (SEQ ID NO: 199) | AK089740 | 8.7 | 117.0 | 159.0 | 993.0 |
| FT332854 (SEQ ID NO: 200) | AK171891 | 17.2 | 247.0 | 343.0 | 2095.0 |
| FT332893 (SEQ ID NO: 201) | AK171928 | 27.4 | 351.0 | 410.0 | 2541.0 |
| FT332945 (SEQ ID NO: 202) | AK156448 | 8.5 | 99.0 | 180.0 | 841.0 |
| FT332952 (SEQ ID NO: 203) | AK171984 | 7.2 | 102.0 | 175.0 | 908.0 |
| FT332964 (SEQ ID NO: 204) | AK171993 | 11.2 | 353.0 | 15.0 | 1106.0 |
| FT333050 (SEQ ID NO: 205) | AK156533 | 12.2 | 199.0 | 157.0 | 501.0 |
| FT333068 (SEQ ID NO: 206) | AK089828 | 7.2 | 58.0 | 65.0 | 201.0 |
| FT333175 (SEQ ID NO: 207) | AK172115 | 7.2 | 96.0 | 109.0 | 586.0 |
| FT333179 (SEQ ID NO: 208) | AK172117 | 8.2 | 155.0 | 235.0 | 1310.0 |
| FT333197 (SEQ ID NO: 209) | AK156724 | 6.2 | 80.0 | 83.0 | 234.0 |
| FT333198 (SEQ ID NO: 210) | AK089889 | 4.2 | 24.0 | 57.0 | 234.0 |
| FT333211 (SEQ ID NO: 211) | AK156768 | 8.2 | 100.0 | 193.0 | 868.0 |
| FT333243 (SEQ ID NO: 212) | AK156805 | 11.2 | 151.0 | 176.0 | 1129.0 |
| FT333321 (SEQ ID NO: 213) | AK172236 | 8.0 | 37.8 | 129.0 | 413.0 |
| FT333323 (SEQ ID NO: 214) | AK172239 | 8.7 | 36.3 | 55.0 | 273.0 |
| FT333388 (SEQ ID NO: 215) | AK172311 | 9.2 | 144.0 | 181.0 | 1006.0 |
| FT333481 (SEQ ID NO: 216) | AK172445 | 4.0 | 28.4 | 45.6 | 203.0 |
| FT333657 (SEQ ID NO: 217) | AK172626 | 13.2 | 186.0 | 273.0 | 1327.0 |
| FT333902 (SEQ ID NO: 218) | AK147712 | 13.2 | 153.0 | 14.2 | 480.0 |
| FT333907 (SEQ ID NO: 219) | AK147745 | 8.7 | 303.0 | 8.7 | 1073.0 |
| FT334029 (SEQ ID NO: 220) | AK144215 | 14.7 | 220.0 | 17.2 | 701.0 |
| FT334036 (SEQ ID NO: 221) | AK090117 | 19.2 | 246.0 | 20.2 | 734.0 |
| FT334049 (SEQ ID NO: 222) | AK149633 | 7.5 | 258.0 | 10.0 | 899.0 |
| FT334695 (SEQ ID NO: 223) | AK146302 | 14.2 | 165.0 | 14.2 | 511.0 |
| FT334704 (SEQ ID NO: 224) | AK146406 | 11.2 | 186.0 | 14.2 | 637.0 |
| FT334792 (SEQ ID NO: 225) | AK146672 | 13.7 | 116.0 | 15.2 | 342.0 |
| FT34247 (SEQ ID NO: 226) | AK012536 | 9.7 | 138.0 | 15.2 | 462.0 |
| FT35741 (SEQ ID NO: 227) | AK132600 | 8.2 | 71.0 | 12.2 | 214.0 |
| FT39889 (SEQ ID NO: 228) | AK134682 | 3.2 | 82.0 | 3.7 | 300.0 |
| LIT1703 (SEQ ID NO: 229) | M63671 | 966.5 | 1357.5 | 1357.5 | 2727.8 |
| LIT1742 (SEQ ID NO: 230) | AY035343 | 3.7 | 2.3 | 4.1 | 3315.4 |
| LIT1755 (SEQ ID NO: 231) | AF323987 | 5.0 | 3.2 | 6.6 | 521.8 |
| LIT1756 (SEQ ID NO: 232) | AF357456 | 8.3 | 1.0 | 5.9 | 657.7 |
| LIT3461 (SEQ ID NO: 233) | DQ059756 | 551.1 | 451.3 | 521.8 | 2816.6 |
| RF00017 (SEQ ID NO: 234) | AC091263 | 79.7 | 150.0 | 112.5 | 365.9 |
| RF00017d (SEQ ID NO: 235) | AC127411 | 214.9 | 241.4 | 287.3 | 706.3 |

In another embodiment, the invention provides a method of detection of TRAIN in tumors and/or normal cells and/or tissues comprising using immunohistochemical assessment of ongoing interferon signaling. In one example, the ongoing interferon signaling is determined by detecting proteins encoded by interferon-responsive genes in a biological sample obtained or derived from an individual.

In another embodiment, the invention provides a method of screening of potentially carcinogenic substances for the ability to induce TRAIN in mammalian cells. In certain aspects, this comprises providing a potentially carcinogenic substance and contacting mammalian cells with the substance, wherein induction of TRAIN in the cells subsequent to being contacted by the substance is indicative that the substance is carcinogenic.

In another embodiment, a method of diagnostics of aging comprising determining indicia of TRAIN in a sample of normal tissue and/or and body fluids obtained or derived from a mammal is provided.

In another embodiment, the invention provides a method of screening and/or identification of agents suitable for treatment and/or prophylaxis of aging and/or cancer by identifying agents with selective cytotoxicity against cells with activated TRAIN. In certain examples, this comprises providing a plurality of agents for testing, and contacting cells with activated TRAIN with the agents, wherein an agent which kills cells with activated TRAIN, but does not kill cells which do not exhibit activated TRAIN, is indicated to be an agent that is suitable for treatment and/or prophylaxis of aging and/or cancer.

Kits for carrying out methods of the invention are also provided. The kits comprise one or more primers and/or probes suitable for use in detecting any combination of the transcripts described herein.

The following specific examples are provided to illustrate the invention, but are not intended to be limiting in any way.

### Example 1

This Example demonstrates that DNA demethylation can be lethal for p53-null, but is not for p53-wild type, cells.

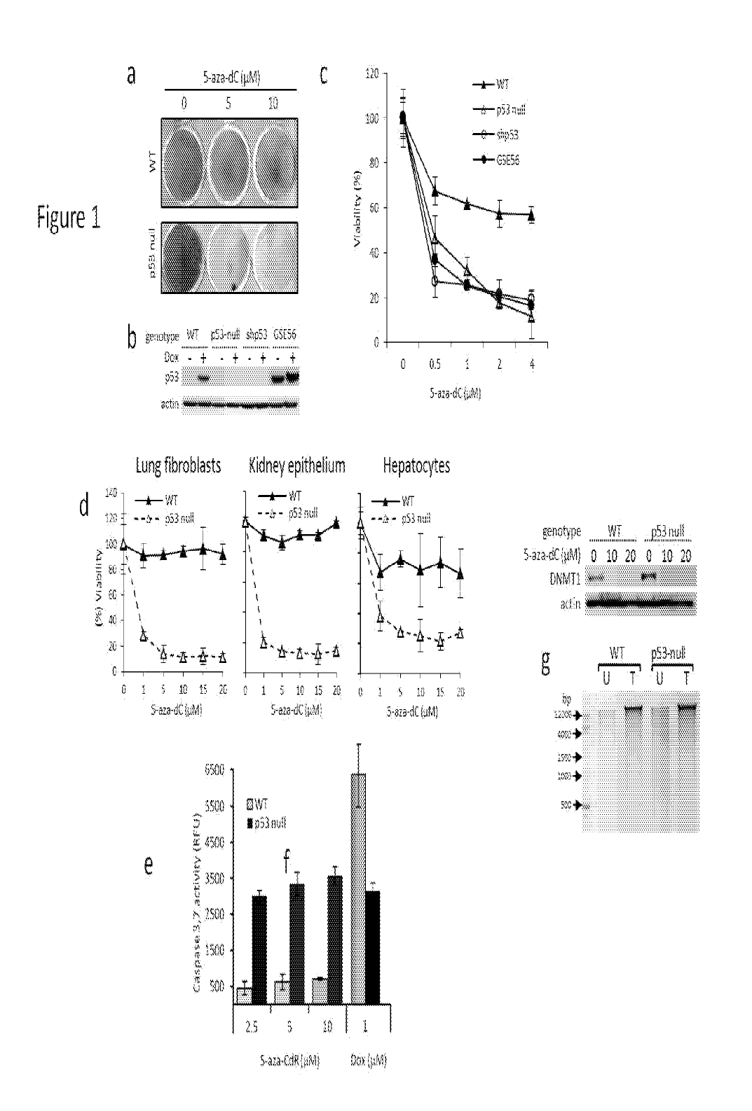

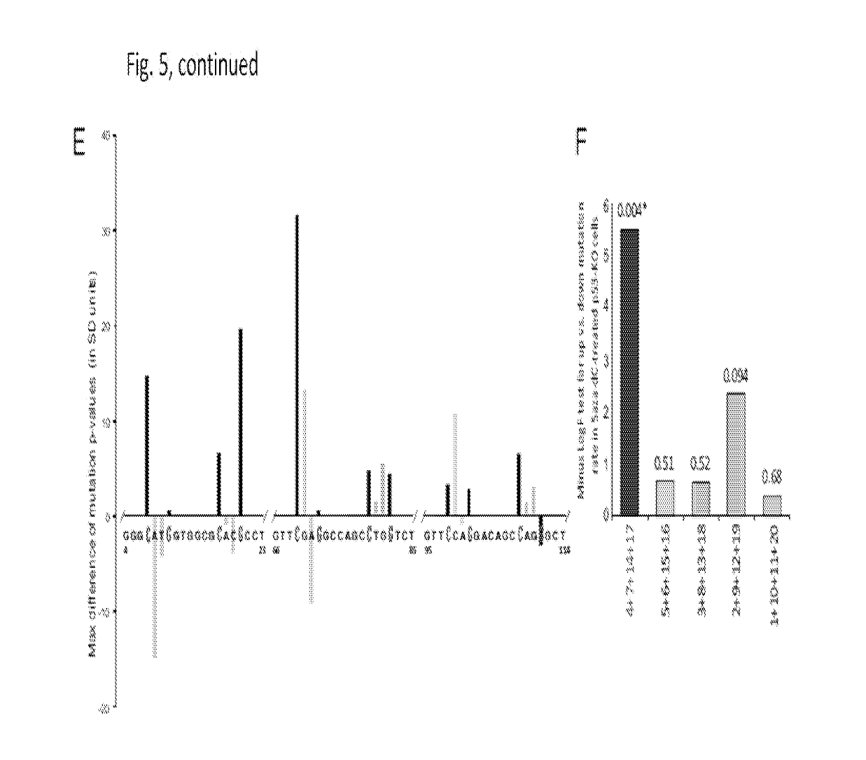

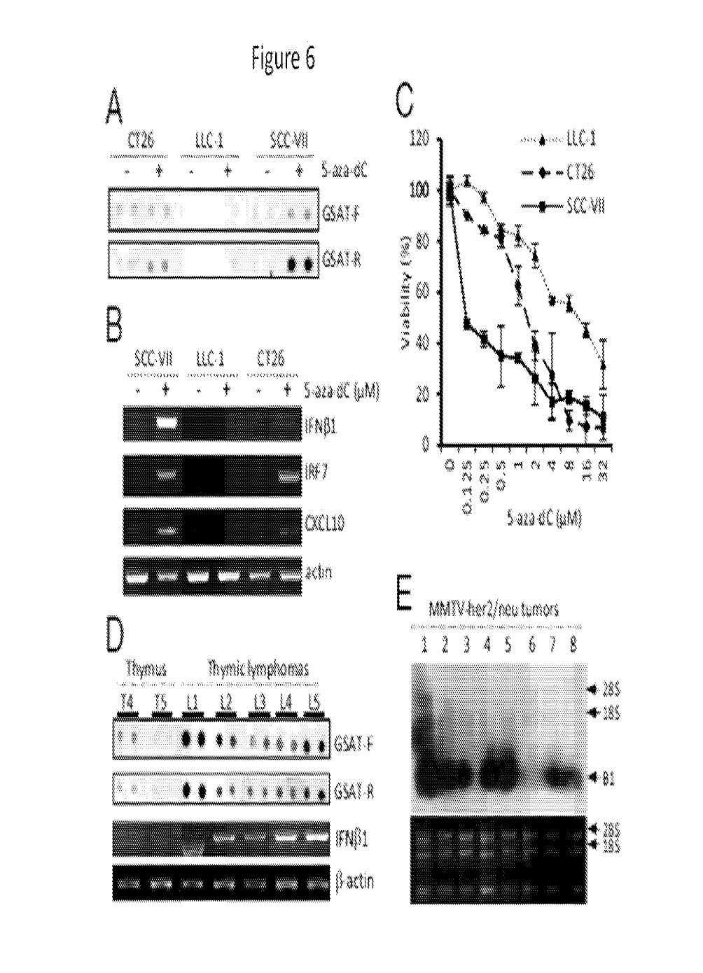

In vitro treatment with the DNA demethylating agent 5-aza-2′-deoxycytidine (5-aza-dC) induced apoptosis in mouse embryonic fibroblasts (MEFs) lacking p53, but not in those with wild type (WT) p53. We extended this observation (FIG. 1A, C;) by showing that MEFs, irrespective of the sex of the embryo (FIG. 7A, 7B), become highly sensitive to 5-aza-dC following either shRNA-mediated knockdown of p53 or ectopic expression of a dominant negative p53 mutant, GSE56 (20) (FIG. 1B, C). The latter result indicates that sensitization to 5-aza-dC does not require physical absence of p53 but only its functional inactivation. High sensitivity to DNA demethylating agent is not limited to p53-deficient fibroblasts and is also the property of fibroblasts and epithelial cells isolated from different tissues of adult mice (FIG. 1D). This phenomenon was observed only in proliferating p53-null cells (FIG. 8) and required at least 72 hours of 5-aza-dC treatment, which reflects the time needed for the compound to fully exert its DNA demethylating activity (19). Death of 5-aza-dC-treated p53-null cells was associated with activation of caspases 3 and 7, indicating involvement of apoptosis (FIG. 1E).

A potential explanation for the observed phenomenon would be that 5-aza-dC is inefficient as a demethylating agent in cells with functional p53 due to their ability to undergo growth arrest following DNA damage (known to be caused by 5-aza-dC (19)). This possibility was ruled out by our findings of (i) complete depletion of free DNA methyltransferase I (DNMT1) protein in both p53-WT and p53-null cells after 5-aza-dC treatment (FIG. 1F), and (ii) similar degrees of DNA demethylation in the genomic DNA of p53-WT and p53-null cells following 5-aza-dC treatment as evidenced by similar patterns of DNA digestion by methylation-sensitive restriction enzyme McrBC (FIG. 1G).

In summary, sensitivity to 5-aza-dC-induced apoptosis is a common property of p53-deficient mouse cells regardless of their tissue of origin or the mechanism of p53 inactivation (knockout, knockdown or functional inactivation). This differentiates 5-aza-dC from other DNA damaging agents, which are generally more toxic to p53-WT cells (21). Indeed, doxorubicin treatment induced higher levels of caspases 3 and 7 in p53-WT than in p53-null MEFs (FIG. 1E). This suggests that the selective toxicity of 5-aza-dC to p53-null cells is likely associated with its demethylating activity rather than its DNA damaging activity.

### Example 2

This Example demonstrates that different subsets of genes are activated by 5-aza-dC in p53-null and p53-WT MEFs.

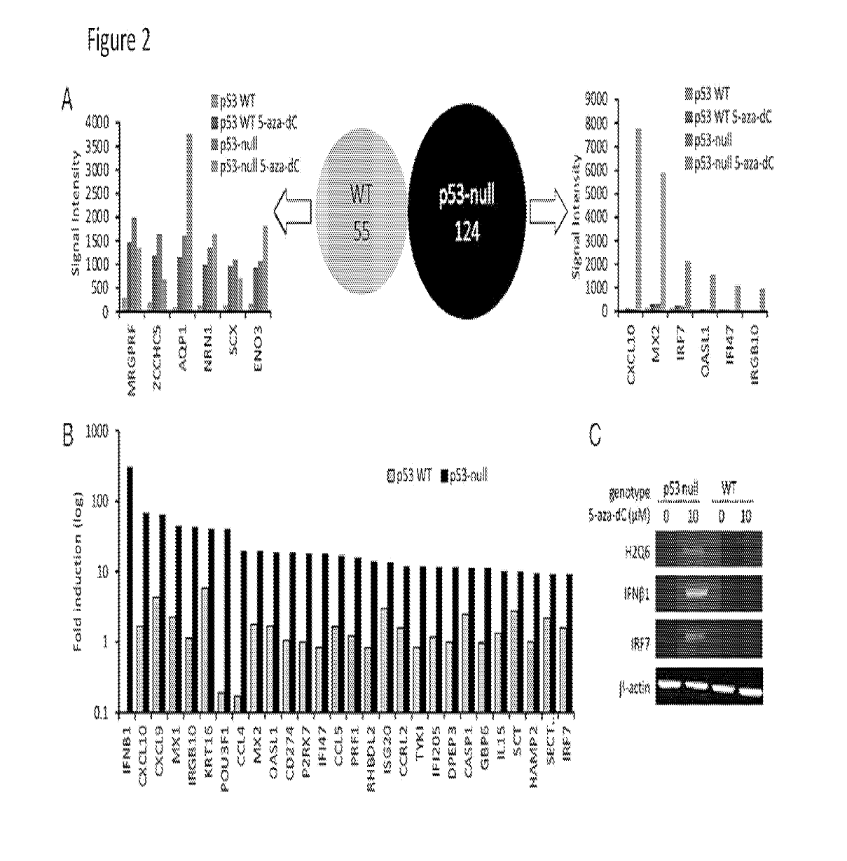

It is believed that many potentially functional genetic elements in mammalian genomes are transcriptionally repressed due to methylation of CpG dinucleotides in the vicinity of their promoters (22). Derepression of some genes regulated in this way (e.g., tumor suppressors p16, PTEN, Arf, etc.) can have a detrimental effect on cell growth or even be cytotoxic (23). Therefore, we hypothesized that the hypersensitivity of p53-null cells to 5-aza-dC-mediated might be due to activation of transcription of some “killer” genes that are normally repressed by the combined action of a DNA methylation-mediated mechanism and the trans-repressor function of p53. To identify these putative “killer” genes, we compared sets of transcripts activated by 5-aza-dC treatment in p53-WT and p53-null MEFs shortly before the onset of the toxic effect of the drug (i.e., at 48 hrs) using microarray (Illumina MouseWG-6 v2.0) hybridization-based global gene expression profiling. The results of this comparison are summarized in FIG. 2.

Treatment of p53-WT and p53-null MEFs with 5-aza-dC led to activation (by at least 5-fold) of 55 and 124 genes, respectively (Table 1 and 1). Surprisingly, there were no genes shared between these two lists (Venn diagram, FIG. 2A). To investigate the reason underlying this, we compared the basal expression levels of genes identified as 5-aza-dC-inducible in either cell line in untreated p53-WT and p53-null MEFs. This showed that for the majority of genes induced by 5-aza-dC in p53-WT MEFs, the induced level of RNA expression only reached the basal level observed in untreated p53-null cells (FIG. 2A-left panel). Therefore, in the absence of p53, these genes are not suppressed by DNA methylation, thus identifying a role for p53 as a driver of DNA methylation-mediated gene silencing.

Transcripts upregulated by 5-aza-dC treatment in p53-null cells demonstrated a completely different pattern of behavior. With a few exceptions, they all remained “silent” (expressed at low/undetectable levels) in p53-WT MEFs regardless of 5-aza-dC treatment, yet were strongly upregulated by 5-aza-dC in p53-null cells (FIG. 2A, right panel). The literature indicated that the vast majority of these genes are known transcriptional targets lying downstream of type I IFNs (IFNα and IFNβ) (Table 2). Moreover, one gene in the group with the highest degree of induction encoded mouse IFNβ1 (FIG. 2B, Table 2). The microarray results were validated using semi-quantitative RT-PCR analysis of several representative genes from the identified subsets (FIG. 2C). Taken together, these data confirm association of p53 deficiency with (i) lack of silencing (high basal expression) of genes that are transcribed in p53-WT cells only after 5-aza-dC treatment, and (ii) induction of a strong IFN response by 5-aza-dC.

### Example 3

This Example demonstrates that DNA demethylation in p53-null cells triggers a lethal IFN response.

Production of type I IFNs is a major anti-viral response that limits the infectivity of a wide range of DNA and RNA viruses (24). In addition to this major function, type I IFNs are also involved in complex interactions with a wide range of biological outcomes, including cytostatic and cytotoxic effects (24). Therefore, since 5-aza-dC induced a strong IFN response in p53-null cells (but not p53-wt), we set out to test the functionality of the induced IFN response and determine whether it is involved in the hypersensitivity of p53-null cells to the drug.

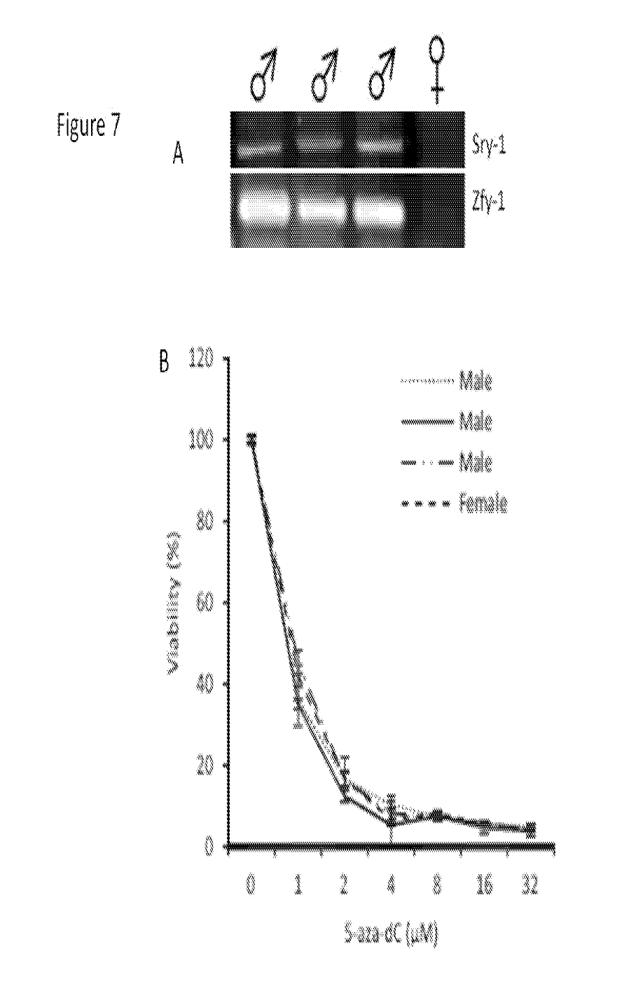

As a measure of the functionality of the IFN response, we assessed the ability of MEFs to support infection with an IFN-sensitive virus, vesicular stomatitis virus (25) (VSV). Treatment with 5-aza-dC strongly repressed virus replication only in p53-null cells (FIG. 9), thus confirming the functionality of the IFN response in these cells.

To determine whether the 5-aza-dC-induced IFN response plays a role in the toxicity of the drug towards p53-null cells, we used MEFs from IFN receptor 1 (ifnar) knockout mice (26), which are incapable of developing a type I IFN response. We introduced into these cells the constructs described above that either knockdown (shRNA) or inactivate (GSE56) p53 (FIG. 3A). While these methods of eliminating p53 activity made p53-WT MEFs with intact IFNAR sensitive to 5-aza-dC-mediated killing, they did not sensitize p53-WT, ifnar^{-/-} MEFs (FIG. 3B, C). No signs of caspase activation were detected in ifnar^{-/-} MEFs treated with 5-aza-dC (FIG. 3D). These observations suggest that the IFN response is a major mediator of death induced by 5-aza-dC in p53-deficient cells.

Since DNA demethylation is expected to induce new transcripts and IFN response can be triggered by virus-like (i.e. double-stranded) RNA species, we tested the IFN-inducing capacity of RNA isolated from p53-WT and p53-null cells, untreated or treated with 5-aza-dC. After removal of rRNA (to reduce high background levels of abundant and largely double-stranded and abundant RNA species) RNA samples were then transfected into SCCVII cells (a mouse head-and-neck cancer-derived cell line known to retain normal type I IFN response) and expression of the IFN-responsive protein p49 (27) was evaluated by Western blotting. Transfection of RNA isolated from 5-aza-dC-treated p53-null cells resulted in dramatically higher induction of p49 expression as compared to RNA from 5-aza-dC-treated p53-WT cells or untreated cells of either genotype (FIG. 3E). This result suggests the presence of an IFN-inducing RNA species specifically in p53-null cells with reduced DNA methylation.

### Example 4

This Example demonstrates massive transcription of repetitive elements in p53-null cells treated with 5-aza-dC.

IFN responses are normally triggered by double-stranded RNA (dsRNA) interpreted by the cell as an indication of viral infection (24). The gene expression profiling that we performed using the Illumina mousewG-6 v2.0 microarray assayed only protein-coding mRNA transcripts and did not reveal any candidate triggers for the suicidal IFN response observed in 5-aza-dC-treated p53-null cells. Therefore, we used a high-throughput RNA sequencing technique to build a comprehensive unbiased picture of RNA species induced by 5-aza-dC treatment. Total RNA was isolated from p53-WT and p53-null MEFs left untreated or treated for 48 hours with 5-aza-dC. After depletion of rRNA, the RNA samples were fragmented, converted to double-stranded cDNA using random primers, ligated with adaptors, and sequenced using a HiSeq2000 instrument (Illumina) with approximately 80×10⁶ 50-nucleotide reads per sample.

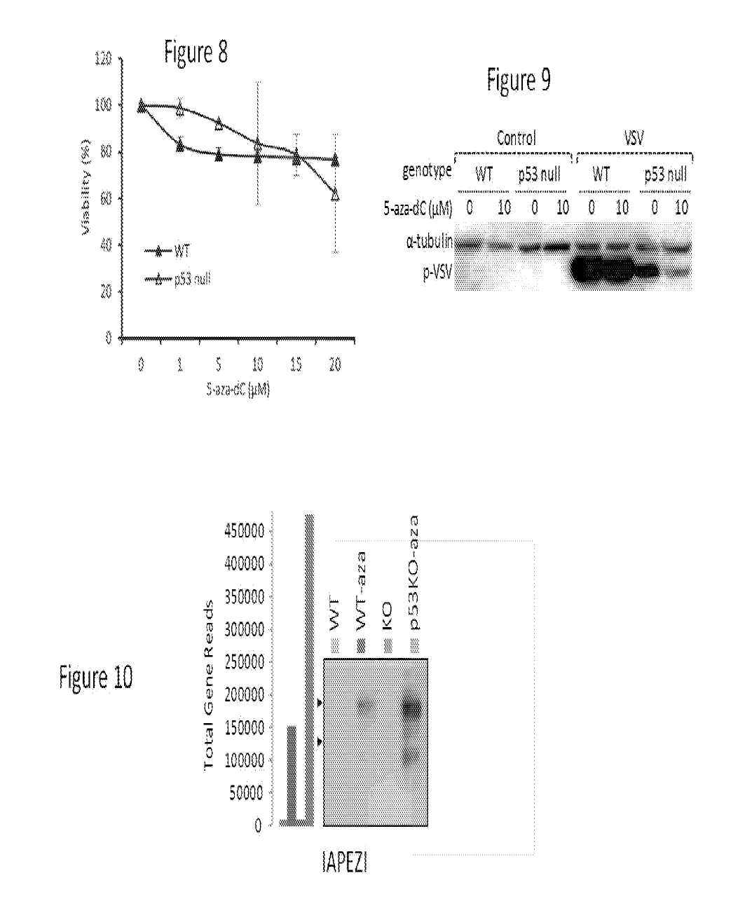

The sequencing results were analyzed by assigning individual cDNA sequences to specific entries in mammalian genome databases that include, besides protein-coding transcripts, functional RNA species (rRNA, snRNA, tRNA, etc.), RNAs transcribed from various classes of repeats, and non-coding RNAs (28). The majority of database entries were equally represented among the four RNA samples; however, three types of RNA transcripts were specifically and significantly more abundant in the RNA sample from 5-aza-dC-treated p53-null MEFs. These included: (i) both major classes of mouse SINEs, namely B1 and B2 (FIG. 4B, C) (ii) near-centromeric major (gamma) satellite repeats (GSAT) (FIG. 4A), and (iii) a large number of different non-coding RNA species (ncRNAs) (FIG. 4D and Table 3). Importantly, we also identified repeats and ncRNAs that were induced in both p53-WT and p53-null MEFs upon treatment with 5-aza-dC, albeit to a greater extent in p53-null cells. These transcripts apparently represent cases of methylation-based silencing that only partially depends on p53 and included, for example, transcripts of endogenous retrotransposons such as intracisternal A-particles (IAP) (FIG. 10), “primitive” relatives of retroviruses present in mouse genomes at about 10,000 copies (29). All of these observations were confirmed using Northern blot hybridization with independently isolated RNA samples from untreated and 5-aza-dC-treated p53-WT and p53-null MEFs (FIG. 4A-D and FIG. 10).

Northern blotting demonstrated that both 5-aza-dC-treated and untreated p53-WT cells as well as untreated p53-null cells expressed sequences corresponding to B1 SINE predominantly within large RNA transcripts, while RNA corresponding to individually transcribed B1 elements (116 bp long RNA-polymerase III-driven transcript) was barely detectable (FIG. 4C). However, in the sample from 5-aza-dC-treated p53-null MEFs, the most intense signal was concentrated in the size range of single-element transcripts, thus indicating that DNA demethylation in the absence of p53 results in strong activation of transcription of B1 and B2 elements. Annealing of these induced monomeric positive strands of B1 and B2 transcripts to antisense-oriented B1 and B2 sequences present in the untranslated regions of numerous protein-coding mRNAs can be predicted to create a large pool of dsRNA potentially capable of triggering an IFN response (FIG. 4F).

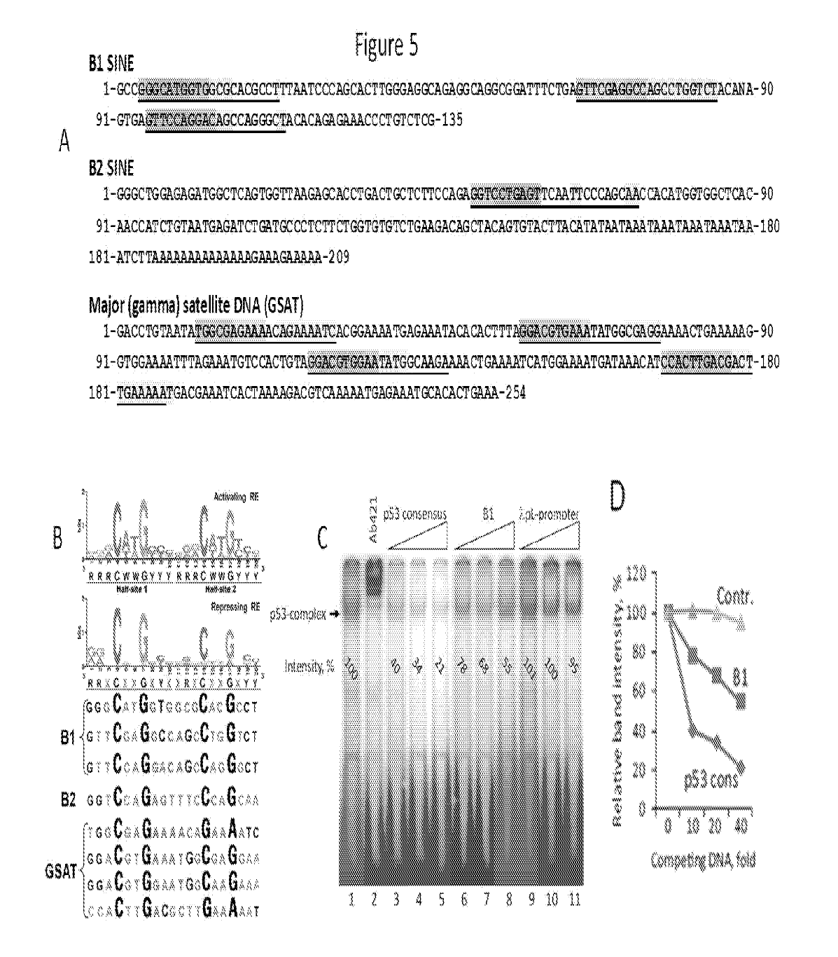

In contrast to SINEs, transcripts of GSAT sequences vary in length from 240 bp to more than 10 kb (see Northern blot in FIG. 4A). Detection of GSAT transcripts can be revealed by radiolabeled probes representing both positive and negative strands of GSAT DNA (FIG. 6A, D) indicative of bi-directional transcription. This is consistent with previous reports in which temporary activation of GSAT transcription and appearance of dsRNA of various lengths was observed during specific stages of embryonic development (30) and in the hearts of old adult mice (31). dsRNA formed by annealed complementary GSAT RNA strands could trigger IFN induction.

To gain an appreciation of the potential biological impact of the identified 5-aza-dC-induced transcripts in p53-null MEFs, we estimated their overall abundance vis-à-vis the abundance of mRNAs for β-actin, a highly expressed transcript commonly used as a reference in gene expression studies. We calculated the relative representation of RNAs transcribed from SINEs (B1 and B2), satellite DNA (GSAT and SATMIN), IAPs and ncRNAs in each sample based on the proportion of their corresponding sequences in RNA samples used for high throughput sequencing. The results shown in FIG. 4E demonstrate that the amount of new RNA (counted as numbers of monomeric copies) synthesized specifically in p53-null cells following treatment with 5-aza-dC was more than 150 times greater than the level of β-actin mRNA. Two-thirds of this new pool of RNA was comprised of transcripts of SINEs and satellite DNA in near equal proportions and the remaining one-third consisted of equal proportions of IAP transcripts and ncRNAs. Interestingly, however, IAPs were not among the RNA species that showed differential expression between untreated p53-null and untreated p53-WT MEFs, thus suggesting that their DNA methylation-based silencing is not strictly p53-dependent.

### Example 6

This Example demonstrates structural properties of major classes of p53-controlled repeats.

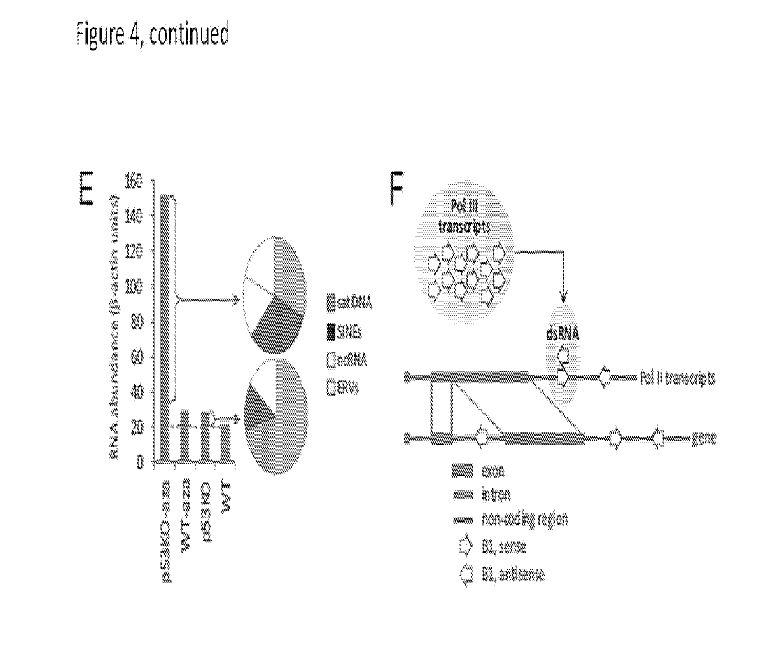

Reconstruction of the phylogenetic history of SINEs suggests that their amplification in mammalian genomes started about 65 million years ago and involved a series of “explosions” that created subfamilies of repeats each with shared mutations (9, 12, 32). To determine whether B1 and B2 transcription initiated by DNA hypomethylation in p53-null cells involved random or specific subsets of repeats, we calculated deviation from the consensus SINE sequence (built by analyzing the entire SINE family in the mouse genome) for each nucleotide position of the B1 and B2 sequences identified in sequencing of our four RNA samples (p53-WT and p53-null MEFs, untreated and 5-aza-dC-treated). No major differences were found in the profiles of nucleotide polymorphisms along B1 and B2 transcripts among all four samples indicative of roughly random activation of transcription of different subsets of SINEs in p53-null cells treated with 5-aza-dC (correlation coefficients>0.96). However, more precise analysis revealed significant shifts in frequencies of mutations in a number of positions in B1 transcripts in the latter RNA sample versus three others. Interestingly, nucleotide substitutions were significantly less frequent in specific positions within putative p53-binding sequences (see FIG. 5E, F).

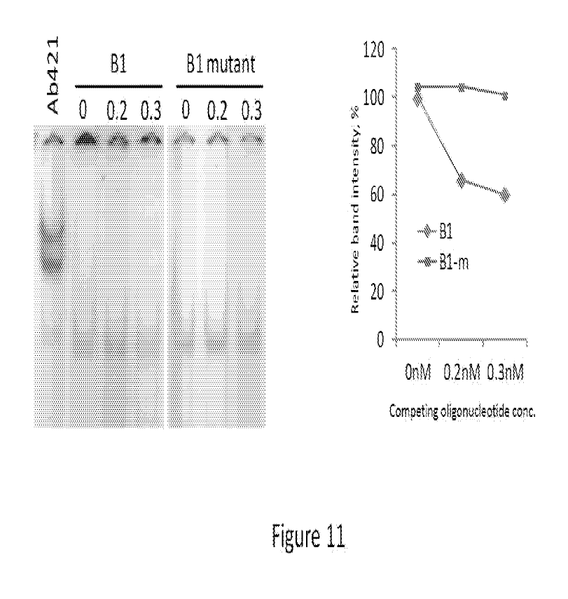

p53 is known as a repressor of transcription acting via recognition of specific sequences that are similar to those that serve as p53 binding sites in p53-induced genes (33, 34). A search for putative p53 binding-like sites revealed a series of candidate sequences in B1, B2 and GSAT elements (FIG. 5A, B). GSAT elements were found to be especially enriched with potential p53-binding sequences. The functionality of predicted p53 binding sites was tested using EMSA with synthetic oligonucleotides representing different B1 fragments incubated with nuclear extracts of mouse cells known to have wild type and functional p53. While the oligonucleotides used did not show stable binding with p53 under the applied conditions, they nevertheless were capable of inhibiting p53 binding to a control oligonucleotide containing a consensus p53-binding site (FIG. 5 C, D). This inhibition was dependent on the presence of intact putative p53 binding sites in B1-derived oligonucleotides since point mutations at key nucleotides of the p53-binding sequence completely abrogated the ability of oligonucleotide to compete for p53 binding (FIG. 11). Remarkably, the mutation frequencies in these particular nucleotides were less frequent than in the rest of the positions within p53-binding sites of new B1 transcripts induced by 5-aza-dC treatment (FIG. 5E, F). These observations suggest that B1 repeats are capable of specific p53 binding, albeit with low affinity, and this binding is required for their transcriptional repression by p53.

### Example 7

This Example demonstrates that transcription of repeats and ncRNAs can occur in tumors.

p53 is the most frequently mutated gene in tumors. However, even in tumors that retain wild type p53 gene sequences, p53 is commonly inactivated by other means (e.g., viral protein expression, overexpression of the natural p53 inhibitor mdm2, loss of Arf (35)). Hence, we hypothesized that tumor cells might be prone to induction of transcription of repeats and ncRNAs following 5-aza-dC treatment as was observed in p53-null MEFs. To test this hypothesis, we chose three mouse tumor-derived cell lines (SCC-VII, CT26, and LLC) without significant levels of basal repetitive element transcription (as illustrated by low levels of GSAT transcripts, FIG. 6A). Upon treatment with 5-aza-dC, we found that SCC-VII, CT26 and LLC cells showed strong, intermediate and undetectable expression of GSAT RNA, respectively (FIG. 6A). In addition, we detected strong upregulation of the mRNAs encoding IFNβ1 and its downstream responsive genes IFN regulatory factor 7 (IRF7) and CXCL10 in 5-aza-dC treated SCC-VII cells and, to a somewhat lesser extent, 5-aza-dC treated CT26 cells (FIG. 6B). In contrast, none of these IFN response-associated transcripts was detected in 5-aza-dC-treated LLC cells. Consistent with these findings, SCC-VII cells were the most 5-aza-dC-sensitive cell line (LD50=0.125 μM), CT26 cells showed intermediate sensitivity (LD50=2 μM), and LLC cells were the least sensitive (LD50=14 μM) (FIG. 6C). The identified positive correlation between transcription of repeats, induction of an IFN response, and sensitivity to 5-aza-dC supports our model of tumors with reduced p53 function (36, 37, 38) being prone to activation of transcription of repeats under conditions of reduced DNA methylation. These results suggest that the sensitivity of tumor cells with silent repeats to 5-aza-dC (drug Decitabine approved for treatment of myelodysplastic syndrome and considered for anticancer use) may depend on whether they have functional p53 or not.

Decreased genome-wide DNA methylation is another common property of tumors acquired during in vivo growth (39). Together with inactivation of p53, this could create conditions sufficient to induce transcription of repeats and ncRNAs normally suppressed by a combination of p53 and DNA methylation. Therefore, we hypothesized that transcription of repeats leading to induction of an IFN response might occur spontaneously during tumor growth and progression in vivo. We tested this hypothesis in two mouse tumor models. First, we showed that all tested spontaneous thymic lymphomas, the most frequent tumors to develop in cancer-prone p53-null mice (40), contained much higher levels of repetitive element transcripts than normal tissues of p53-null mice, including thymuses of p53-null mice prior to lymphoma development (FIG. 6D, shown for GSAT RNA transcribed from both DNA strands). As in p53-null MEFs treated with 5-aza-dC, activation of transcription of repeats correlated with the induction of IFN response, as demonstrated by RT-PCR results (FIG. 6D).

Similarly, repetitive element expression was observed in more than half (6/8) of the mammary gland tumors that developed spontaneously in untreated MMTV-her2/neu transgenic mice (41) (FIG. 6D). Previous reports indicate acquisition of missense mutations in at least 37% of these tumors (42). Note that while expression of only one specific class of repeats is shown for each of the above cases, activation of GSAT, B1 and B2 always occurred together. Our finding that growing tumors express high levels of repeats capable of generating dsRNA and subsequently inducing a lethal IFN response suggest that such tumors have successfully passed through selection for resistance to endogenous IFN toxicity.

### CITATIONS FOR EXAMPLE 1-7

1. Makalowski W (2000) Genomic scrap yard: how genomes utilize all that junk. Gene 259:61-67.
2. Kramerov D A, Vassetzky N S (2011) SINEs. Wiley Interdiscip Rev RNA 2:772-786.
3. Smit A F (1999) Interspersed repeats and other mementos of transposable elements in mammalian genomes. Curr Opin Genetics Dev 9:657-663.
4. Ostertag E M, Kazazian H H Jr (2001) Biology of Mammalian L1 Retrotransposons. Annu Rev Genet 35:501-538.
5. Charlesworth B, Sniegowski P (1994) The evolutionary dynamics of repetitive DNA in eukaryotes. Nature 371:215-220.
6. Plohl M, Luchetti A, Mestrovio N, Mantovani B (2008) Satellite DNAs between selfishness and functionality: structure, genomics and evolution of tandem repeats in centromeric (hetero)chromatin. Gene 409:72-82.
7. Quentin Y (1989) Successive Waves of Fixation of B1 Variants in Rodent Lineage History. J Mol Evol 28:299-305.
8. Deininger P L, Batzer M A (2002) Mammalian retroelements. Genome Res 12:1455-1465.
9. Amariglio N, Rechavi G (1993) Insertional mutagenesis by transposable elements in the mammalian genome. Environ Mol Mutagen 21:212-218.
10. Ferrigno O et al (2001) Transposable B2 SINE elements can provide mobile RNA polymerase II promoters. Nat Gen 28:77-81
11. Kramerov D A, Vassetzky NS (2011) Origin and evolution of SINEs in eukaryotic genomes. Nature Her 107:487-495.
12. Carnell A N, Goodman J I (2003) The long (LINEs) and the short (SINEs) of it: altered methylation as a precursor to toxicity. Toxicol Sci 75:229-235.
13. Rhee I et al (2002) DNMT1 and DNMT3b cooperate to silence genes in human cancer cells. Nature 416:552-556.
14. Liu W M, Chu W M, Choudary P V, Schmid C W (1995) Cell stress and translational inhibitors transiently increase the abundance of mammalian SINE transcripts. Nucleic Acids Res 23:1758-1765.
15. Allen T A, Kaenel S V, Goodrich J A, Kugel J F (2004) The SINE-encoded mouse B2 RNA represses mRNA transcription in response to heat shock. Nat Struct Biol 11:816-821.
16. Jang K L, Latchman D S (1992) The herpes simplex virus immediate-early protein ICP27 stimulates the transcription of cellular Alu repeated sequences by increasing the activity of transcription factor TFIIIC. Biochem J 284:667-673.
17. Hagan C R, Rudin C M (2007) DNA Cleavage and Trp53 Differentially Affect SINE Transcription. Genes Chromosom Cancer 46:248-260.
18. Ting D T et al. (2011) Aberrant overexpression of satellite repeats in pancreatic and other epithelial cancers. Science 331:593-596.
19. Nieto M, et al. (2004) The absence of p53 is critical for the induction of apoptosis by 5-aza-2′-deoxycytidine. Oncogene 23:735-743.
20. Ossovskaya V S, et al. (1996) Use of genetic suppressor elements to dissect distinct biological effects of separate p53 domains. Proc Natl Acad Sci USA 93:10309-10314.
21. Bunz F, et al. (1999) Disruption of p53 in human cancer cells alters the responses to therapeutic agents. J Clin Invest 104:263-269.
22. Yoo C B, Jones P A (2006) Epigenetic therapy of cancer: past, present and future. Nature Rev Drug Discov 5:37-50.
23. Jones P A, Baylin S B (2002) The fundamental role of epigenetic events in cancer. Nature Rev Genet 3:415-428.
24. Borden E C, et al. (2007) Interferons at age 50: past, current and future impact on biomedicine. Nature Rev Drug Discov 6:975-990.
25. Liu S-Y, Sanchez D J, Aliyari R, Lu S, Cheng G (2012) Systematic identification of type I and type II interferon-induced antiviral factors. Proc Natl Acad Sci USA 109:4239-4244.
26. Müller U, et al. (1994) Functional role of type I and type II interferons in antiviral defense. Science 264:1918-1921.
27. Fensterl V, White C L, Yamashita M and Sen G C (2008) Novel characteristics of the function and induction of murine p56 family proteins. J Virol 82:11045-11053.
28. Wang Z, Gerstein M, Snyder M (2009) RNA-Seq: a revolutionary tool for transcriptomics. Nature Rev Genet 10:57-63.
29. Barbot W, Dupressoir A, Lazar V, Heidmann T (2002) Epigenetic regulation of an IAP retrotransposon in the aging mouse: progressive demethylation and de-silencing of the element by its repetitive induction. Nucleic Acids Res 40:2365-2373.
30. Probst A V, et al. (2010) A strand-specific burst in transcription of pericentric satellites is required for chromocenter formation and early mouse development. Dev Cell 19:625-638.
31. Gaubatz J W, Cutler R G (1990) Mouse satellite DNA is transcribed in senescent cardiac muscle. J Biol Chem 265:17753-17758.
32. Kidwell M G, Lisch D R (2001) Perspective: transposable elements, parasitic DNA, and genome evolution. Evolution 55:1-24.
33. Ho J, Benchimol S (2003) Transcriptional repression mediated by the p53 tumour suppressor. Cell Death Differ 10:404-408.
34. Wang B, Xiao Z, Ren E C (2009) Redefining the p53 response element. Proc Natl Acad Sci USA 106:14373-14378.
35. Vogelstein B, Lane D, Levine A J (2000) Surfing the p53 network. Nature 408:307-310.
36. Hauser U et al (2002) Reliable detection of p53 aberrations in squamous cell carcinomas of the head and neck requires transcript analysis of the entire coding region. Head & Neck 858-873
37. Langlois M J et al (2010) The PTEN phosphatase controls intestinal epithelial cell polarity and barrier function: role in colorectal cancer progression. Plos One 5:1-12
38. Burdelya L G et al (2006) Inhibition of p53 response in tumor stroma improves efficacy of anticancer treatment by increasing antiangiogenic effects of chemotherapy and radiotherapy in mice. Cancer Res 66:9356-9361.
39. Smet C D, Loriot A (2010) Epigenetic scars of a neoplastic journey DNA hypomethylation in cancer. Epigenetics 5:206-213.
40. Donehower L A, et al. (1992) Mice deficient for p53 are developmentally normal but susceptible to spontaneous tumours. Nature 356:215-221.
41. Green J E, Hudson T (2005) The promise of genetically engineered mice for cancer prevention studies. Nature Rev 5:184-198.
42. Li B, Rosen J M, McMenamin-Balano J, Muller W J, Perkins A S. (1997) neu/ERBB2 cooperates with p53-172H during mammary tumorigenesis in transgenic mice. Mol Cell Biol. 17:3155-63.
43. Lane D P (1992) p53, guardian of the genome. Nature 358:15-16.
44. Lerat E, Sémon M (2007) Influence of the transposable element neighborhood on human gene expression in normal and tumor tissues. Gene 396:303-311.
45. Li T H, Kim C, Rubin C M, Schmid C W (2000) K562 cells implicate increased chromatin accessibility in Alu transcriptional activation. Nucleic Acids Res 28:3031-3039.
46. Missiaglia E, Donadelli M, Palmieri M, Crnogorac-Jurcevic T, Scarpa A, Lemoine N R (2005) Growth delay of human pancreatic cancer cells by methylase inhibitor 5-aza-2′-deoxycytidine treatment is associated with activation of the interferon signalling pathway. Oncogene (24):199-211.
47. Komarova E A, et al., (1997) Transgenic mice with p53-responsive lacZ: p53 activity varies dramatically during normal development and determines radiation and drug sensitivity in vivo. EMBO J 16(6):1391-400.
48. Gottlieb E, et al., (1997) Transgenic mouse model for studying the transcriptional activity of the p53 protein: age- and tissue-dependent changes in radiation-induced activation during embryogenesis. EMBO J 16(6): 1381-1390.
49. MacCallum D E, et al., (1996) The p53 response to ionising radiation in adult and developing murine tissues. Oncogene 13(12): 2575-87.
50. Riley T, Sontag E, Chen P, Levine A J (2008) Transcriptional control of human p53-regulated genes. Nature Rev Mol Cell Biol 9:402-12.
51. Esteve P O, Chin H G, Pradhan S (2005) Human maintenance DNA (cytosine-5)-methyltransferase and p53 modulate expression of p53-repressed promoters. Proc Natl Acad Sci USA 102:1000-1005.
52. Wang Y A, et al. (2005) DNA methyltransferase-3a interacts with p53 and Represses p53-mediated gene expression. Cancer Biol Ther 4:1138-1143.
53. Cui F, Sirotin M V, Zhurkin V B (2011) Impact of Alu repeats on the evolution of human p53 binding sites. Biol Direct 6:2.
54. Beck C R, Garcia-Perez J L, Badge R M, Moran J V. LINE-1 elements in structural variation and disease. Annu Rev Genomics Hum Genet. 2011 Sep. 22; 12:187-215.
55. Li B, Rosen J M, McMenamin-Balano J, Muller W J, Perkins A S. neu/ERBB2 cooperates with p53-172H during mammary tumorigenesis in transgenic mice. Mol Cell Biol. 1997 June; 17(6):3155-63.
56. Nozawa J et al (1999) Loss of transcription factor IRF-1 affects tumor susceptibility in mice carrying the Ha-ras transgene or nullizygosity for p53. Genes Dev 13:1240-1245.
57. Haus O. The genes of interferons and interferon-related factors: localization and relationships with chromosome aberrations in cancer. Arch Immunol Ther Exp (Warsz). 2000; 48(2):95-100.
58. Heyman M, Grander D, Brondum-Nielsen K, Cederblad B, Liu Y, Xu B, Einhorn S. (1994) Interferon system defects in malignant T-cells. Leukemia. (8): 425-34
59. Fountain J W, Karayiorgou M, Ernstoff M S, Kirkwood J M, Vlock D R, Titus-Ernstoff L, Bouchard B, Vijayasaradhi S, Houghton A N, Lahti J, (1992) Homozygous deletions within human chromosome band 9p21 in melanoma. Proc Natl Acad Sci USA., (89): 10557-10561.
60. Lydiatt, W. M., Davidson, B. J., Schantz, S. P., Caruana, S. and Chaganti. (1998) R S. 9p21 deletion correlates with recurrence in head and neck cancer. Head Neck., (20):113-118.
61. Ring C J A (2002) Cytolytic viruses as potential anti-cancer agents. J Gen Virol 83:491-502.
62. Langmead B, Trapnell C, Pop M, Salzberg S L (2009) Ultrafast and memory-efficient alignment of short DNA sequences to the human genome. Genome Biol 10:R25.

### Example 8

This Example provides a description of the materials and methods used to obtain the results described in the previous Examples.

Analysis of Results of Total RNA Sequencing

Mapping of Illumina reads onto repetitive elements and ncRNA species was performed by the BOWTIE program (62) under default parameters. As to non-coding RNAs, the sequences from all the RNAdb (http://research.imb.uq.edu.au/madb/) sub-bases (fantom 3, RNAz, etc) were used for mapping of reads from the samples. The Repbase database (http://www.girinst.org/repbase/) was used as a set of unique repetitive elements for the mapping. The expression level of each ncRNA and repetitive element was calculated as median per-position coverage of its sequence by mapped reads. This is the per-position normalized measure of expression that does not depend on the sequence length. In order to give a biological dimension to this measure of expression for the studied ncRNA species and repetitive elements, each value was re-calculated in “β-actin units”. Namely, first, the reads of four RNA-sequence samples were mapped onto β-actin mRNA, and the median per-position counts of this mRNA across samples were calculated. Next, the per-position measure of expression for a particular ncRNA or a repetitive element in a sample was divided by the per-position value of expression for β-actin in the same sample, which gives the expression level of each studied sequence across the four RNA-seq samples in units of the β-actin expression in the corresponding sample.

Mapping of reads from the RNA samples onto B1 and B2 consensus sequences was performed by a blast-like alignment program that detects mutations more accurately than the Burrows-Wheeler indexing based programs. For each position, a significance of the mutation in three treated samples regarding the WT sample was calculated via Poisson statistics. Namely, for each position of the consensus, an expected number of counts of the consensus nucleotide at this position in a treated sample were calculated based on a frequency of this nucleotide in the WT sample. A distance in standard deviation (SD) units between the real number of counts of the consensus nucleotide in a treated sample, and expected number of nucleotide counts defines polymorphism of this particular position. The 20 SDs limit was taken as a mutation significance threshold.

Cell Lines, Chemicals and Reagents

5-aza-2′-deoxycytidine and doxorubicin were purchased from Sigma. Unless otherwise specified, all drug treatments were carried out in Dulbecco's modified Eagle's medium (DMEM) (Invitrogen) supplemented with 10% Fetal bovine serum (FBS) and 50 ug/ml penicillin/streptomycin (Gibco). Mouse melanoma B16, squamous cell carcinoma SCC-VII, and Lewis-lung carcinoma LLC-1 cell lines were purchased from American Type Culture Collection (ATCC) and maintained in DMEM with 10% FBS. Mouse colon tumor CT26 cells were purchased from ATCC and maintained in Roswell Park Memorial Institute medium (RPMI) with 10% FBS and 50 ug/ml penicillin/streptomycin. Green fluorescent protein-tagged vesicular stomatitis virus (VSV-G/GFP) was a kind gift of Dr. Ganes Sen (Cleveland Clinic, Cleveland Ohio). Antibiotics for selection of cells transduced with retroviral/lentiviral expression vectors (see below) were puromycin (Invitrogen) and G418 (Gibco).

Mice

A colony of p53 wild-type and p53-knockout (1) mice on a C57BL/6 background was maintained by crossing p53_{+/-} females with p53_{-/-} males (purchased from Jackson Laboratories, Bar Harbor, Me.) followed by PCR based genotyping of the progeny. A colony of ifnar_{-/-} deficient (2) mice on C57BL/6 background was maintained in Cleveland Clinic (Cleveland Ohio).

Embryo Gender Determination

The gender of individual p53-null embryos was determined as described (3). Briefly, genomic DNA from each embryo was isolated using the PureLink™ Genomic DNA Kit (Invitrogen) and amplified by PCR (30 cycles) with primers for SRY (sense 5′-GATCAGCAAGCAGCTGGGATACCAGTG-3′ (SEQ ID NO:236), and antisense 5′-CTGTAGCGGTCCCGTTGCTGCGGTG-3′) (SEQ ID NO:237), and ZFY (sense: 5′-CCTATTGCATGGACTGCAGCTTATG-3′ (SEQ ID NO: 238), 5′-GACTAGACATGTCTTAACATCTGTC-3′(SEQ ID NO: 239), both located on the Y chromosome.

Primary Culture of Mouse Embryonic Fibroblasts (MEF)

MEFs were isolated from 13.5-days post-coitus embryos by breeding C57BL/6 mice p53-heterozygous (p53_{+/-}) females and males as described in the protocol (http://www.molgen.mpg.de/˜rodent/MEF_protocol.pdf). The embryos were individually plated and DNA was isolated from each embryo using the PureLink™ Genomic DNA

Kit (Invitrogen). The genotype of the embryos was determined by PCR with the following primers: 5′-ACAGCGTGGTGGTACCTTAT-3′ (SEQ ID NO: 240); 5′-TATACTCAGAGCCGGCCT-3′ (SEQ ID NO: 241); 5′-TCCTCGTGCTTTACGGTATC-3′ (SEQ ID NO: 242). MEFs were cultured in DMEM supplemented with 10% FBS and 50 ug/ml penicillin/streptomycin for no more than 8 passages.

Primary Cultures of Cells from Adult Lung and Kidney

Primary cell cultures were established with cells isolated from lung and kidney tissue of 8-week old C57BL/6 p53^{+/-} and 53^{-/-} mice. Briefly, tissues were finely minced and incubated in DMEM media containing 2 mg/ml dispase (Gibco) at 4° C. for 1 hour in a 50 ml Falcon tube. The mixture was then mechanically dissociated and placed at 37° C. for 30 min. This step was repeated twice more, each time adding 2 mg/ml of freshly prepared dispase enzyme. Up to 50 ml of DMEM was added to the tube before centrifugation at 10,000×rpm for 5 minutes. The supernatant was removed and cells were plated in DMEM supplemented with 10% FBS and 50 ug/ml penicillin/streptomycin.

Primary Culture of Adult Mouse Hepatocytes

Hepatocytes were isolated from adult male C57BL/6 p53-wt and p53-null mice after two-step perfusion (performed essentially as described (4) with some modifications). Briefly, liver perfusion was performed on mice deeply anesthetized with Isoflurane through the inferior vena cava using a peristaltic pump at a speed of 3-4 ml per minute. The liver was perfused with EGTA (0.5 mM EGTA (Sigma) in PBS without calcium and magnesium, 30 ml) following collagenase (0.02% collagenase (Sigma) in DMEM medium containing penicillin/streptomycin, 50 ml). After perfusion, the gall bladder was removed; the liver was dissected from the animal and gently disrupted in 50 ml DMEM. Cells were filtered through a cell strainer and left to sediment for 20 min at +4° C. The cell pellet was dissolved in 20 ml DMEM. 10 ml of cell suspension was overlaid on the top of a two-step Percoll (Fisher Scientific) gradient (50% and 25% Percoll in PBS, 20 ml each) in a 50 ml conical tube (two tubes per liver). Purified hepatocytes were collected from the bottom of the tube after centrifugation (20 min, 1750×g) and resuspended in 50 ml DMEM plus 10% FCS. The typical yield was 40-50×10⁶ nuclei per liver (note that the majority of adult mouse hepatocytes are bi-nuclear) with 93-95% viability. After attachment (usually 1-1.5 hours) plates were washed, medium was replaced with William's E medium (WEM) (Invitrogen) containing 10% FCS and supplements (penicillin/streptomycin, 2 mM glutamine (Invitrogen), 10 mM nicotinamide (Sigma), ITS (Sigma), 50 ng/ml EGF (Peprotech) and 10-7M dexamethasone (Sigma)) (5). Quiescent hepatocytes were kept in DMEM medium supplemented with 10% FBS.

Cell Viability Assay

To determine cell viability, cells were plated in triplicate in 96-well plates at a density of 3,000 cells per well and treated with increasing concentrations of 5-aza-dC as specified in the text. At the indicated time, cells were stained using 0.4% methylene blue (USB) in 50% methanol (BDH). Plates were photographed or the dye was extracted from stained cells using 3% HCl solution for spectrophotometric quantitation.

Estimation of 5-Methylcytosine Content in Genomic DNA

Genomic DNA was extracted from p53-WT and p53-null MEFs left untreated or treated with 10 μM 5-aza-dC for 48 hours using the PureLink™ Genomic DNA Kit (Invitrogen) according to the manufacturer's instructions. DNA samples (1 μg) were digested with the methylation-sensitive McrBC restriction enzyme (New England Biolabs) and resolved on a 1.5% agarose gel.

Western Immunoblotting

Protein extracts were prepared by lysing cells in RIPA buffer (Sigma) with protease inhibitor cocktail (Sigma). Extracts were spun down at 10,000 rpm for 10 minutes at 4° C. to obtain the soluble fraction. Protein concentrations were determined by BioRad Protein Assay (Bio-Rad). Equal amounts of protein were run on gradient 4-20% precast gels (Invitrogen) and blotted/transferred to Immobilon-P membrane (Millipore). Membranes were blocked with 5% non-fat milk-TBS-T buffer for 1 hour and incubated overnight with primary antibodies. The following primary antibodies were used: Anti-p53 (Ab-1) (Pantropic) Mouse mAb (PAb421) (Calbiochem); anti-p-VSV (a kind gift of Dr. Amiya Banerji, Cleveland Clinic); and anti-dnmt-1 mouse mAb (Calbiochem). To verify equal protein loading and transfer, β-actin (Santa Cruz) or α-tubulin (Santa Cruz) antibodies were used. Anti-mouse and anti-rabbit secondary horseradish peroxidase-conjugated antibodies were purchased from Santa Cruz. ECL detection reagent (GE Healthcare) was used for protein visualization on autoradiography film (Denville Scientific).

Detection of Interferon Induction by Western Immunoblotting

Total RNA was depleted of its ribosomal fraction by using Ribo-Zero Ribosomal RNA removal kit (Epicentre) following manufacturer's instructions. SCC-VII cells were transfected by calcium phosphate method with 500 ng of total RNA, 500 ng of ribosomal depleted RNA or 1 μg/ml of poly I:C (Sigma) along with 250 ng of plv-CMV-GFP plasmid as control. Protein extracts were prepared 29 hours later by lysing cells in RIPA buffer (Sigma) with protease inhibitor cocktail (Sigma). Extracts were spun down at 10,000×rpm for 10 minutes at 4° C. to obtain the soluble fraction. Protein concentrations were determined by BioRad Protein Assay (Bio-Rad). Equal amounts of protein were run on gradient 4-20% precast gels (Invitrogen) and blotted/transferred to Immobilon-P membrane (Millipore). Membranes were blocked with 5% non-fat milk-TBS-T buffer for 1 hour and incubated overnight with primary antibodies. The following antibodies were used: mouse p49 (a kind gift of Dr. Ganes C. Sen, Cleveland Clinic). To verify equal protein loading and transfer β-actin (Santa Cruz) antibody was used. Anti-rabbit secondary horseradish peroxidase-conjugated antibody was purchased from Santa Cruz. ECL detection reagent (GE Healthcare) was used from protein visualization onto Autoradiography film (Denville Scientific).

High Throughput Sequencing of Total Poly(A)+ RNA

Total RNA was isolated from p53-WT and p53-null MEFs left untreated or treated with 5-aza-dC (10 μM for 48 hours) using Trizol (Life Technologies) according to the manufacturer's protocol. Total RNA samples (5 μg) were depleted of 28S, 18S and 5S rRNA using the Ribo-Zero rRNA removal kit (Agilent) according to the manufacturer's protocol. rRNA-depleted total RNAs were converted to adaptor-ligated cDNA using the reagents and protocol of the TruSeq RNA Sample Preparation kit (Illumina) with minor modifications. Briefly, rRNA-depleted total RNAs were fragmented to 200-300 nt fragments by incubation in Elute/Prime/Fragment solution (Illumina) at 98° C. for 8 min, reverse transcribed with HP MMLV-RT (Agilent) in 50-p1 reaction mix at 25° C. for 10 min and 42° C. for 1 hr, followed by second-strand cDNA synthesis in 100-ul reaction mix using second-strand master mix (Illumina, mixture of DNA polymerase I and RNAse H) at 16° C. for 1 hr and double-stranded cDNAs were purified by Qiaquick PCR purification kit (Qiagen). Double-stranded cDNAs were treated with end repair mix (Illumina, mix of Klenov enzyme and T4 DNA polymerases) at 30° C. for 30 min, purified using Qiaquick purification kit (Qiagen), tailed with 3′-A-overhangs using A-tailing mix (Illumina, mix of Klenow exo-DNA polymerase and rATP) at 37° C. for 30 min, ligated with RNA adapter 5 (Illumina) using ligation mix (Illumina, T4 DNA ligase) at 30° C. for 30 min and purified by Qiaquick PCR purification kit (Qiagen). Adapter ligated cDNAs were amplified by Titanium DNA polymerase (Clontech) in 100 μl of PCR reaction buffer using PCR primer mix (Illumina) and the following PCR cycling conditions: 10 cycles of (94° C. for 30 sec, 60° C. for 10 sec and 72° C. for 30 sec). Amplified cDNA products were purified by Qiaquick PCR purification kit (Qiagen) and separated in preparative 3.5% agarose/1×TAE gel. PCR products with sizes 180-300 bp (cover appr. 90% of amplified size distribution) were excised from agarose gel, purified by Qiaquick Gel Extraction Kit (Qiagen), quantitated by OD at 260 nm, adjusted to 10 nM concentrations. The final adaptor-ligated cDNA samples were sequenced in HiSeq2000 (Illumina) single read flow cells using the HP6 sequencing primer (50 nt reads and approximately 80×10₆ reads per sample).

Vesicular Stomatitis Virus (VSV) Infection

p53-WT and p53-null MEFs were plated at equal densities and either left untreated or treated with 10 μM 5-aza-dC for 36 hours in DMEM supplemented with 10% FBS. Equal titers of VSV-G/GFP virus was added for 6 hours, after which the presence of the virus was assayed by Western blotting with antibodies specific to the P-protein of the virus.

Electrophoretic Mobility Shift Assay (EMSA)

Nuclear extracts were isolated from doxorubicin-treated B16 cells as described (6, 7). Each binding reaction contained 10 mM HEPES (pH 7.5), 80 mM KCl, 1 mM EDTA, 1 mM EGTA. 6% glycerol, 0.5 μg of sonicated salmon sperm DNA, 5 μg of nuclear protein extract and a 5′-end′-[³²P]-labeled double stranded oligonucleotide probe containing the p53 binding sequence from the p21 (WAF1) promoter (5′-AGCTTAGGCATGTCTAGGCATGTATA-3′ (SEQ ID NO:275); putative p53 binding sites located within the mouse SINE B1 sequence: (5′-GCCGGGCATGGTGGCGCACGCCTTTAATCCCAGCACTTGGGAAGAGGCAGGCGG ATTTCTGAGTTCGAGGCCAGCCTGGTCTAC-3′ (SEQ ID NO:243); point mutations introduced into the putative p53-binding sites located within SINE B1 sequence: 5′-GCCGGGAATTGTGGCGAACTCCTTTAATCCCAGCACTTGGGAGGCAGAGGCAGG CGGATTTCTGAGTTAGATGCCAGCATGTTCTAC-3′ (SEQ ID NO: 244). DNA-binding reactions were carried out at room temperature for 30 minutes. To determine sequence specificity anti-p53 (pab421) (Calbiochem) antibodies were added to the reaction. The cold oligonucleotide competitor challenge was carried out by incubating DNA-binding reactions with increasing excesses of unlabeled oligonucleotide containing the SINE B1 ‘p53-binding’ site sequence (400 nM (1:10), 800 nM (1:20), and 1600 nM (1:40)). DNA-protein complexes were separated on a 6% acrylamide gel and visualized by Typhoon Phospho-Imager.

Polymerase Chain Reaction

cDNA was synthesized from 1 μg of total RNA isolated by TRIzol (Invitrogen) using the iScript cDNA Synthesis Kit (BioRad). PCR was carried according to the manufacturer's protocol with Taq PCR Master Mix (USB) in a reaction volume of 25 μl with 100 ng of the following primers: mouse-IFNβ1 (sense: 5′-CTCCACGCTGCGTTCCTGCT-3 (SEQ ID NO: 245); antisense: 5′-TCGGACCACCATCCAGGCGT-3′ (SEQ ID NO: 246), mouse-IRF7 (sense: 5′-CAGCCAGCTCTCACCGAGCG-3′ (SEQ ID NO: 247); antisense: 5′-GCCGAGACTGCTGCTGTCCA-3′ (SEQ ID NO: 248) mouse-H2-Q6 (sense: 5′-TGTGACGTGGGGTCCGACGA-3′ (SEQ ID NO: 249); antisense: 5′-AGGGTAGAAGCCCAGGGCCC-3′(SEQ ID NO: 250) mouse CXCL10 (sense: 5′-TGGCTAGTCCTAATTGCCCTTGGT-3′(SEQ ID NO: 251), antisense: 5′-TCAGGACCATGGCTTGACCATCAT-3′(SEQ ID NO: 252), mouse-β-actin (sense: 5′-GCTCCGGCATGTGCAA-3′ (SEQ ID NO: 253). antisense: 5′-AGGATCTTCATGAGGTAGT-3′(SEQ ID NO: 254).

Retroviral/Lentiviral Vectors and Transduction

We used retroviral plasmid encoding dominant-negative inhibitor of p53, GSE56 (8) and lentiviral plasmid encoding shRNA against p53 (9) as previously described. For preparation of retroviral stocks, Phoenix-ampho packaging cells were used, while 293T cells were used for preparation of lentiviral stocks. Cells were transfected with the vectors, using Lipofectamine-2000 reagent (Invitrogen); in the case of lentivirus, two helper plasmids, pCMVΔR8.2 and pVSV-G, were cotransfected along with experimental vector. Supernatants containing infectious viral particles were harvested 24, 36, and 48 hours post-transfection, pooled, and filtered. Infections of exponentially growing cells were performed with virus-containing supernatant supplemented with 4 μg/mL polybrene (Sigma).

Microarray Analysis

Total RNA was extracted using Trizol (Invitrogen) from p53-WT and p53-null MEFs that were either untreated or treated with 10 μM 5-aza-dC for 48 hours. The resulting four RNA samples were analyzed on Illumina Mouse WG-6 v2.0 Expression BeadChips containing probes for more than 45,200 transcripts by the Gene Expression Facility at Roswell Park Cancer Institute (Buffalo, N.Y.).

Northern Hybridization

Mouse cDNA probes for SINE B1 (sense: 5′-GCCTTTAATCCCAGCACTTG-3′ (SEQ ID NO: 255), antisense: 5′-

CTCTGTGTAGCCCTGGTCGT-3′ (SEQ ID NO: 256), SINE B2 (sense: 5′-GCACCTGACTGCTCTTCCAGAGGT-3′(SEQ ID NO: 257), antisense: 5′-TCTTCAGACACACCAGAAGAGGGCA-3′(SEQ ID NO: 258), IAPEZI (sense: 5′-TGGATGGGCTCGGGAAGCCA-3′(SEQ ID NO: 259), antisense: 5′-TCTCTGCGCACTGCTGACGC-3′(SEQ ID NO: 260), FR0149069 (sense: 5′-GCCGCTCGGCACTCCTTTGT-3′(SEQ ID NO: 261), antisense 5′-CCTGCCGACTGGCAAACGGT-3′(SEQ ID NO: 262), FR0228915 (sense: 5′-GGTCGCTGGCCACTCAGCTC-3′ (SEQ ID NO: 263) antisense: 5′-TAACGGCGAATGTGGGGGCG-3′(SEQ ID NO: 264), and gamma-satellite (sense: 5′-TGGCGAGGAAAACTGAAAAAGGTGG-3′(SEQ ID NO: 265), antisense: 5′-GCCATATTCCACGTCCTACAGTGGA-3′(SEQ ID NO:266), were generated by RT-PCR from total RNA of MEF cells. The cDNAs were labeled with [α³²P]-dCTP using the Random Primed DNA Labeling Kit following the protocol provided by Roche (Mannheim, Germany). Total RNA was extracted from p53-WT and p53-null MEFs that were either untreated or treated with 10 μM 5-aza-dC for 48 hours using Trizol (Invitrogen). Total RNA (5 μg per lane) was electrophoresed in an agarose-formaldehyde gel and transferred to Hybond-N membrane (Amersham Pharmacia Biotech). After UV crosslinking, membranes were hybridized with [032P]-dCTP-labeled probes and analyzed by autoradiography at −80° C. To determine SINE B1 expression in mouse spontaneous tumors, MMTV/her2neu tumor-bearing mice were sacrificed to excise tumors. Total RNA was extracted using Trizol (Invitrogen) and Northern blotting was performed as described above.

Dot-Blot Hybridization

Single stranded oligonucleotide probes for gamma-satellite forward [(F): 5′-

CTGTAGGACGTGGAATATGGCAAGAA-3′(SEQ ID NO:267)] and reverse [(R): 5′-TTTCTCATTTTTGACGTCTTTTAGTGA-3′(SEQ ID NO:268)] strands were 5′-end′-[α³²P]-labeled using T4 polynucleotide kinase (Promega). Unincorporated radioactivity was removed using Illustra MicroSpin-G-25 Columns (GE Life Sciences). Total RNA was isolated using Trizol (Invitrogen) from cells (CT26, LLC-1, SCC-VII) left untreated or treated with 10 μM 5-aza-dC for 48 hours. Total RNA was also isolated from tumor-free thymuses or tumor-bearing thymuses of p53-null C57BL/6 mice using Trizol. Total RNA was denatured by boiling for 5 min and blotted (500 ng per dot) onto a positively charged nylon membrane (GE Life Sciences). After UV crosslinking, membranes were prehybridized at 58° C. for 1 hour and then hybridized at 58° C. overnight with [³²P]-labeled probe. The membranes were analyzed by autoradiography at −80° C.

Caspase Activity Assay

The caspase 3,7 activation assay was performed using 30 mM of fluorigenic Ac-DEVD-AMC substrate (ENZO). Cells were incubated in buffer [50 mM HEPES (pH 7.5), 100 mM NaCl, 2 mM EDTA, 0.1% CHAPS, 10% sucrose and 5 mM DTT] with 30 nM Ac-DEVD-AMC at 37° C. for 1 hour. Caspase activity was quantified by spectrophotometer (excitation at 380 nm and emission at 440 nm).

### REFERENCES FOR MATERIALS AND METHODS

1. Donehower L A et al (1992) Mice deficient for p53 are developmentally normal but susceptible to spontaneous tumours. Nature 356:215-221.
2. Müller U. et al. (1994) Functional role of type I and type II interferons in antiviral defense. Science 264:1918-1921.
3. Koopman P, Gubbay J, Vivian N, Goodfellow P, Lovell-Badge R (1991) Male development of chromosomally female mice transgenic for Sry. Nature 351:117-121.
4. Gleiberman A S, Sharovskaya YuYu, Chailakhjan L M (1989) “Contact inhibition” of alpha-fetoprotein synthesis and junctional communication in adult mouse hepatocyte culture. Exp Cell Res 184:228-234.
5. Fougère-Deschatrette C., et al. (2006) Plasticity of hepatic cell differentiation: bipotential adult mouse liver clonal cell lines competent to differentiate in vitro and in vivo. Stem Cells 24: 2098-2109.
6. Lyakh L A, et al. (2000) Bacterial lipopolysaccharide, TNF-alpha, and calcium ionophore under serum-free conditions promote rapid dendritic cell-like differentiation in CD14+ monocytes through distinct pathways that activate NK-kappa B. J Immunol 165:3647-3655.
7. Gasparian A V, et al. (2011) Curaxins: anticancer compounds that simultaneously suppress NF-κB and activate p53 by targeting FACT. Sci Transl Med 3:95-74.
8. Ossovskaya V S, et al. (1996) Use of genetic suppressor elements to dissect distinct biological effects of separate p53 domains. Proc Natl Acad Sci USA 93:10309-10314.
9. Boiko A D, et al. (2006) A systematic search for downstream mediators of tumor suppressor function of p53 reveals a major role of BTG2 in suppression of Ras-induced transformation. Genes Dev 20:236-252.

While the invention has been described through specific embodiments, routine modifications will be apparent to those skilled in the art and such modifications are intended to be within the scope of the present invention.

We claim:

1. A method for detection of cancer or a pre-cancerous condition in an individual comprising: detecting in a sample obtained or derived from the individual at least one RNA transcript, the transcription of which is repressed by a combination of functional p53 and DNA methylation in a cell that is not a cancer or a pre-cancerous cell.

2. The method of claim 1, wherein the at least one RNA transcript is transcribed from a repeat of a genomic DNA polynucleotide sequence.

3. The method of claim 1, wherein a plurality of RNA transcripts is detected, and wherein the plurality of transcripts is a combination of at least two of the transcripts selected from the group of transcripts defined in Table 1, Table 2, Table 3, SEQ ID NO:276 through SEQ ID NO:351, and combinations thereof.

4. The method of claim 4, wherein the plurality of RNA transcripts is a combination of the RNA transcripts defined in Table 2.

5. The method of claim 1, wherein the biological sample is a sample comprising blood.

6. A method for predicting sensitivity of a tumor in an individual to treatment with a DNA demethylating agent comprising determining from a biological sample from the individual the presence or absence of:
i) ncRNA and/or RNA transcripts transcribed from a repeat of genomic polynucleotide sequence repeat, the transcription of which is normally suppressed by functional p53, or
ii) constitutive activation of an interferon type I response; or
iii) a combination of i) and ii).

7. The method of claim 6, comprising determining the absence of i), or ii) or iii) and identifying the tumor in the individual as likely to be sensitive to the treatment with the DNA demethylating agent.

8. The method of claim 7, further comprising treating the individual with the DNA demethylating agent.

9. The method of claim 7, wherein the DNA demethylating agent is 5-aza-dC.

10. A method of classification of a tumor, the method comprising determining whether or not the tumor exhibits indicia of TRAIN, wherein the indicia of TRAIN comprise:
i) ncRNA species and/or RNA transcripts transcribed from polynucleotide sequence repeats, the transcription of which is normally suppressed by functional p53, or
ii) constitutive activation of an interferon type I response; or
iii) a combination of i) and ii).

11. The method of claim 10, wherein the plurality of the ncRNA species and the RNA transcripts is a combination of at least two RNA polynucleotides defined in Table 1, Table 2, Table 3, SEQ ID NO:276 through SEQ ID NO:351, and combinations thereof.

12. The method of claim 11, wherein the plurality of ncRNA species and/or the RNA transcripts is a combination of at least two RNA polynucleotides defined in Table 2.

13. A method of determination of functional status of p53 in a cell or tumor, the method comprising determining whether or not the cell or the tumor exhibits indicia of TRAIN, wherein the indicia of TRAIN comprise:
i) ncRNA species and/or RNA transcripts transcribed from polynucleotide sequence repeats, the transcription of which is normally suppressed by functional p53, or
ii) constitutive activation of an interferon type I response; or
iii) a combination of i) and ii), wherein the presence of indicia of TRAIN is indicative that the p53 is not functional.

14. The method of claim 13, wherein the plurality of the ncRNA species and the RNA transcripts is a combination of at least two RNA polynucleotides defined in Table 1 and/or Table 2 and/or Table 3 and/or by SEQ ID NO:276 through SEQ ID NO:351, and combinations thereof.

15. The method of claim 14, wherein the plurality of ncRNA species and/or the RNA transcripts is a combination of at least two RNA polynucleotides defined in Table 2.
